(19) United States
(12) Patent Application Publication
(10) Pub. No.: US 2024/0226208 A9
(43) Pub. Date: Jul. 11, 2024

(54) ARTIFICIAL ONCOLYTIC VIRUSES AND RELATED METHODS

(71) Applicant: Humane Genomics Inc., New York, NY (US)

(72) Inventors: Chad M. Moles, New York, NY (US); Peter E. Weijmarshausen, Bronxville, NY (US)

(21) Appl. No.: 17/919,278

(22) PCT Filed: Apr. 15, 2021
(86) PCT No.: PCT/US2021/027564

§ 371 Date: Oct. 16, 2022

Related U.S. Application Data
(60) Provisional application No. 63/010,670, filed on Apr. 15, 2020.

(51) Int. Cl. A61K 35/766; C07K 14/005; C12N 7/00

(52) U.S. Cl. CPC A61K 35/766; C07K 14/005; C12N 7/00; C12N 2760/20022; C12N 2760/20051; C12N 2760/20232

(57) ABSTRACT

The disclosure provides artificial viral compositions for use in treating cancer or a hyperproliferative disorder in a subject to whom the compositions are administered, as well as to methods of making and using the compositions.

### RELATED APPLICATIONS

This application claims the benefit of U.S. Provisional Application No. 63/010,670, filed on Apr. 15, 2020. The entire teachings of the above application are incorporated herein by reference.

### BACKGROUND OF THE INVENTION

According to the American Cancer Society, in 2019 there were an estimated 1,762,450 new cancer cases diagnosed and 606,880 cancer deaths in the United States. The treatment of cancer has progressed as understanding of the underlying biological processes has increased. However most current treatment options, including surgery, radiation, chemotherapy, immunotherapy, and newer targeted therapies, continue to be deployed relatively late in cancer development and have undesirable side effects even if successful in addressing the cancer.

Solid tumors present particular therapeutic challenges; for example, cells of a solid tumor do not all present the same mix of antigens on their surfaces, complicating targeting strategies. Additionally, solid tumors can comprise masses of cells thousands of layers thick, making it difficult for therapeutic agents to infiltrate the tumor mass before being eradicated by the patient's immune system. Some solid tumors also produce immune-suppressing agents such as the checkpoint molecule PD-L1, which hamper strategies that enlist the host immune system in killing the tumor cells. Accordingly, treatments having increased effectiveness, precision, impact on quality of life and survivability are still needed.

### SUMMARY OF THE INVENTION

The inventions described herein relate to artificial oncolytic viruses for use in the treatment of cancer, as well as to methods of making these viruses and methods of treating cancer using them.

In one embodiment the invention relates to an artificial oncolytic virus comprising a domain engineered to selectively bind to a target cell via a binding partner identified as highly expressed or preferentially expressed on the target cell as compared with a non-target cell. In some embodiments the binding partner is both highly expressed and preferentially expressed on the target cell as compared with a non-target cell. In some embodiments the binding partner is specifically expressed by the target cell. In some embodiments the engineered domain is encoded by a heterologous DNA sequence. In some embodiments the domain comprises all or a functional portion of a glycoprotein. In some embodiments the subject is Canis lupus familiaris or Homo sapiens.

In some embodiments of the invention expression of the viral genome is engineered to be under the control of a regulatory region of a gene identified as highly expressed or preferentially expressed by the target cell as compared with a non-target cell. In some embodiments the gene is identified as both highly expressed and preferentially expressed by the target cell as compared with a non-target cell. In some embodiments the gene is specifically expressed by the target cell.

In some embodiments of the invention the target cell is a cancer cell (e.g., a tumor cell) or a cell associated with a hyperproliferative disorder. In some embodiments the target cell a cancer cell selected from the group consisting of melanoma, non-small cell lung cancer, small-cell lung cancer, lung cancer, hepatocarcinoma, retinoblastoma, astrocytoma, glioblastoma, gum cancer, tongue cancer, leukemia, neuroblastoma, head cancer, neck cancer, breast cancer, pancreatic cancer, prostate cancer, renal cancer, bone cancer, testicular cancer, ovarian cancer, mesothelioma, cervical cancer, gastrointestinal cancer, lymphoma, brain cancer, colon cancer, and bladder cancer.

In some embodiments the target cell is selected from the group consisting of cells from the bladder, blood, bone, bone marrow, brain, breast, colon, esophagus, gastrointestine, gum, head, kidney, liver, lung, nasopharynx, neck, ovary, prostate, skin, stomach, testis, tongue, and uterus.

In some embodiments of the invention the virus is non-pathogenic in a subject to whom it is intended to be administered. In some embodiments the virus is one to which a subject to whom it is intended to be administered is not immune. In some embodiments the virus is substantially identical to a reference naturally-occurring virus. In some embodiments the virus comprises at least one domain which is identical to a reference naturally-occurring virus. In some embodiments the virus is identical to a reference naturally-occurring virus except for the engineered domain. In some embodiments the virus is identical to a reference naturally-occurring virus except for the engineered regulatory region.

In some embodiments of the invention the reference naturally-occurring virus is a member of the Rhabdoviridae family. In some embodiments the reference naturally-occurring virus is from the genus Vesiculovirus (e.g., Indiana vesiculovirus or New Jersey vesiculovirus).

In some embodiments the virus causes cell death via apoptosis, necrosis, and/or cytopathic effect (CPE) of one or more target cells in the subject upon administration. In some embodiments cell death occurs within 28 days, preferably within 21 days, more preferably within 7 days, more preferably within 2-4 days.

In some embodiments the binding partner is identified using RNAseq data. In some embodiments the gene is identified using RNAseq data. In some embodiments the RNAseq data is obtained from samples from a group of individuals; in other embodiments the RNAseq data is obtained from a database. In certain embodiments the RNAseq data is obtained from an individual subject who is the intended recipient of said virus.

In some embodiments the virus additionally comprises one or more heterologous functional domains selected from the group consisting of a therapeutic agent, a kill switch for said target cell, an agent which facilitates the ability of the virus to evade the recipient immune system, a watermark, a barcode, an agent which degrades the extracellular matrix of a solid tumor, and a diagnostic (e.g., visualization) agent.

The invention also relates to oncolytic viruses comprising the sequence of SEQ ID NO: 2.

The invention also relates to a pharmaceutical composition(s) comprising an artificial oncolytic virus described herein.

The invention further relates to a method of treating a hyperproliferative disorder (e.g., cancer) in a subject (e.g., Canis lupus familiaris or Homo sapiens) comprising administering to the subject a pharmaceutical composition comprising an effective amount of an artificial oncolytic virus described herein. In certain embodiments the cancer is selected from the group consisting of melanoma, non-small cell lung cancer, small-cell lung cancer, lung cancer, hepatocarcinoma, retinoblastoma, astrocytoma, glioblastoma, gum cancer, tongue cancer, leukemia, neuroblastoma, head cancer, neck cancer, breast cancer, pancreatic cancer, prostate cancer, renal cancer, bone cancer, testicular cancer, ovarian cancer, mesothelioma, cervical cancer, gastrointestinal cancer, lymphoma, brain cancer, colon cancer, and bladder cancer.

The invention also encompasses a method of slowing, inhibiting or reducing the growth or size of a tumor comprising administering to the subject (e.g., Canis lupus familiaris or Homo sapiens) a pharmaceutical composition comprising an effective amount of an artificial oncolytic virus described herein. In some embodiments the tumor is selected from the group consisting of non-small cell lung cancer, small-cell lung cancer, lung cancer, hepatocarcinoma, retinoblastoma, astrocytoma, glioblastoma, neuroblastoma, head cancer, neck cancer, breast cancer, pancreatic cancer, prostate cancer, renal cancer, bone cancer, testicular cancer, ovarian cancer, mesothelioma, cervical cancer, gastrointestinal cancer, brain cancer, colon cancer, and bladder cancer.

The invention also relates to a method of producing an artificial oncolytic virus disclosed herein, the method comprising (a) designing the DNA for said virus to prepare viral DNA, wherein the DNA comprises a domain engineered to selectively bind to a target cell via a binding partner identified as highly expressed or preferentially expressed on the target cell as compared with a non-target cell; (b) fragmenting (or defining fragments of) the viral DNA for synthesis; (c) synthesizing the viral DNA fragments; (d) assembling the viral DNA fragments to create artificial oncolytic viral DNA; and (e) transfecting cells with said artificial oncolytic viral DNA to produce artificial oncolytic virus. In certain embodiments design of the DNA is performed using software. In some embodiments design of the DNA is aided by utilization of one or more databases available to the skilled artisan, including, for example, chat.lionproject.net/hallmarks for the hallmarks of cancer; epd.epfl.ch//index.php for promoter selection; oncokb.org/ for identification of targetable binding partner(s); and portals.broadinstitute.org/ccle/page?gene=ERBB2 for gene expression and copy number across various cancer types.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a schematic of oncolytic viral function.

FIG. 2 shows a schematic of oncolytic viral replication.

FIG. 3 shows a schematic of function of an oncolytic virus carrying a therapeutic payload.

FIG. 4 shows a schematic of the use of oncolytic viruses in engineering cancer therapeutics. HGI-001=canine adenovirus (ICOCAV15) expressing immunomodulatory cytokine IL-10; HGI-002=ICOCAV15 expressing IL-10 and CXCR4/CD8 bispecific aptamer; HGI-007=VSV-DsRed; HGI-008=VSV-pCXCR4-anti-PD-1-DsRed; HGI-010=VSV-pCXCR4-anti-PD-1-DsRed; HGI-011=VSV-pCSCR4-anti-PD-L1-DsRed.

FIG. 5 illustrates an overview of this disclosed platform for creating artificial oncolytic viruses.

FIG. 6 shows steps in the creation of artificial viruses of the invention.

FIG. 7 shows a data flow diagram for computer-aided design of personalized artificial oncolytic virotherapies.

FIG. 8 shows real-time PCR results. Significant upregulation and overexpression of ERBB3/HER3 in CMT-12 and CMT-U27 and CXCR4 in CMT-U27 and D17 relative to control sample NCF.

FIG. 9 shows real-time PCR results. Significant upregulation and overexpression of CXCR4 and CMT-U27 in BHK-21 relative to control sample NCF.

FIG. 10 provides a table providing examples of the core components that may be utilized during the design and manufacture of an oncolytic virus described herein.

FIG. 11 provides a schematic of a fully synthetic VSV genome driven by a T7 system, featuring HH and HDV ribozyme.

FIG. 12 provides a schematic of a fully synthetic VSV with a moxGFP transgene driven by a CMV promoter.

FIG. 13 provides a schematic of a fully synthetic VSV genome having the glycoproteins of VSV replaced with the glycoproteins of SVV.

FIG. 14 demonstrates live-cell imaging validates functionality of modular VSV genome design and capacity to deliver transgene of interest. GFP images (10× magnification) depict cells only (control), VSV wildtype, and VSV encoding a moxGFP transgene driven by a CMV promoter at 24 hours post-transfection.

FIG. 15 provides a phase image (10× magnification) of VSV-SVV causing CPE at 24 hours post-transfection in BHK-21.

FIG. 16 provides a schematic of a promoter-reporter-polyA [promoter-sfGFP-BGH].

FIG. 17 demonstrates canine synthetic promoters are functional and exhibit predictable behavior. Cell lines of interest (OSCA-8, OSCA-32, OSCA-78, and D22) were seeded in 48-well plates, monitored for 24 hours, and then transfected with DNA fragments encoding a cancer specific promoter and reporter (sfGFP). Putative promoter sequences for COL5A3, VEGFA, ERBB2/HER2, ANGPTL2, OMD, IGF1R, MYC, COL3A1, and HIF1A were designed and included. Fluorescence intensity was measured for control wells (cells only) to determine background fluorescence and set GFP threshold gates for analysis. Green fluorescence intensity (a.u.) was then measured and normalized to cell area per image to determine promoter activity. CMV was used as a control to gauge promoter activity and strength.

FIG. 18 demonstrates human synthetic promoters are functional and exhibit predicable behavior. Cell lines of interest (U2-OS, HOS) were seeded in 48-well plates, monitored for 24 hours, and then transfected with DNA fragments encoding a cancer specific promoter and reporter (sfGFP). Haman synthetic promoters were evaluated in U2-OS and HOS, including putative sequences for ERBB2/HER2, VEGFA, FGFR1, HAS1, HIF1A, MYC, MKI67, and IL6. Fluorescence intensity was measured for control wells (cells only) to determine background fluorescence and set GFP threshold gates for analysis. Green fluorescence intensity (a.u.) was then measured and normalized to cell area per image to determine promoter activity. EF1A was used as a control promoter to assess strength and activity of putative designs.

FIG. 19 demonstrates promoter designs are functional and predictable. RNA was extracted from D22 using a TaqMan Cells-to-CT kit (Thermo Fisher). Real-time PCR reactions were conducted with Fast Advanced Master Mix (Applied Biosystems) using a QuantStudio 6 Flex (Applied Biosystems). Reaction and cycling conditions were performed according to the manufacturer's recommendations. TaqMan assays (Applied Biosystems) for COL3A1, MYC, ERBB2, and IGF1R were used to determine gene expression. The CT mean for each gene was normalized to a reference gene (GAPDH) activity and plotted on the x-axis. Promoter assay data (fluorescence measurements background subtracted and normalized to CMV) was plotted on the y-axis.

FIG. 20 demonstrates cancer specific promoters display high selectivity and validate design methods. D22 (canine osteosarcoma cell line) and NCF (healthy canine cells) were seeded in a 48-well plate, incubated for 24 hours, and then transfected in triplicate with DNA fragments encoding cancer specific promoters and reporter genes (mKate2). HD Phase and RFP images (n=3 images/well) were acquired at 10× magnification after 24 hours. Fluorescence intensity was measured for control wells (cells only) to determine background fluorescence and set RFP threshold gates for analysis. Red fluorescence intensity (a.u.) was then measured and normalized to cell area per image to determine promoter activity. Values were then normalized to fluorescent output generated by a strong mammalian promoter (CMV).

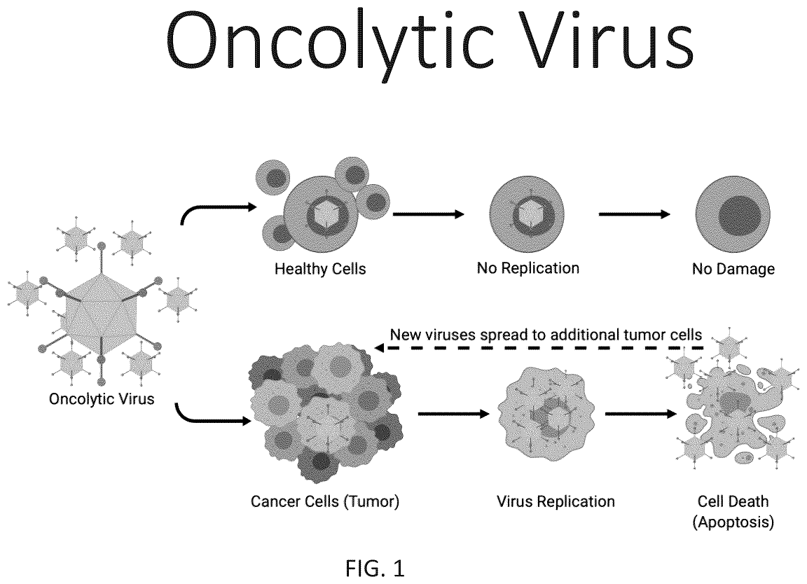

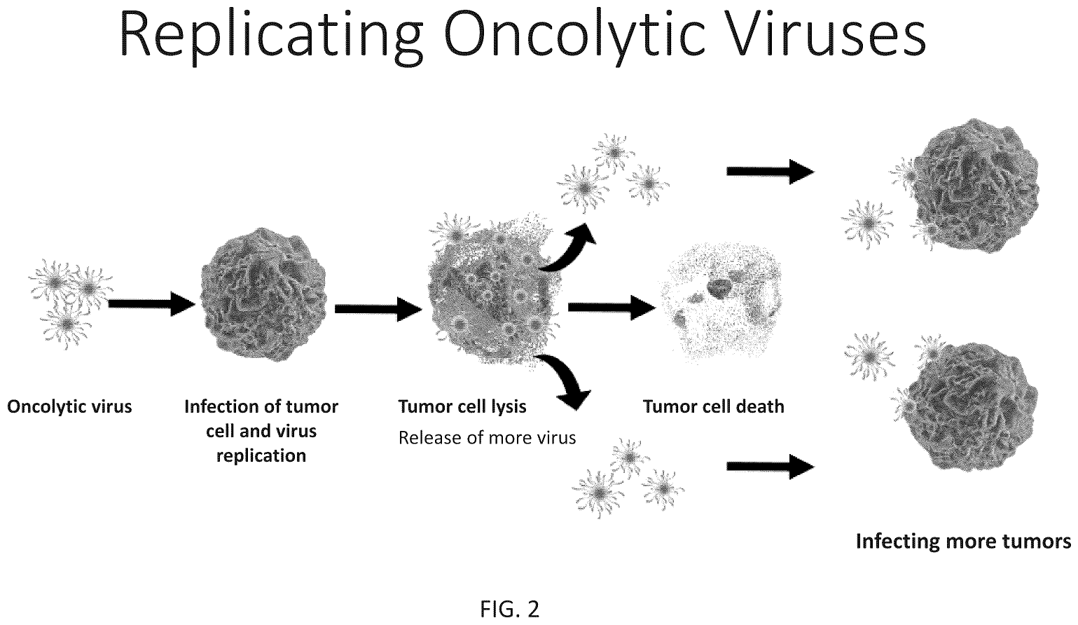

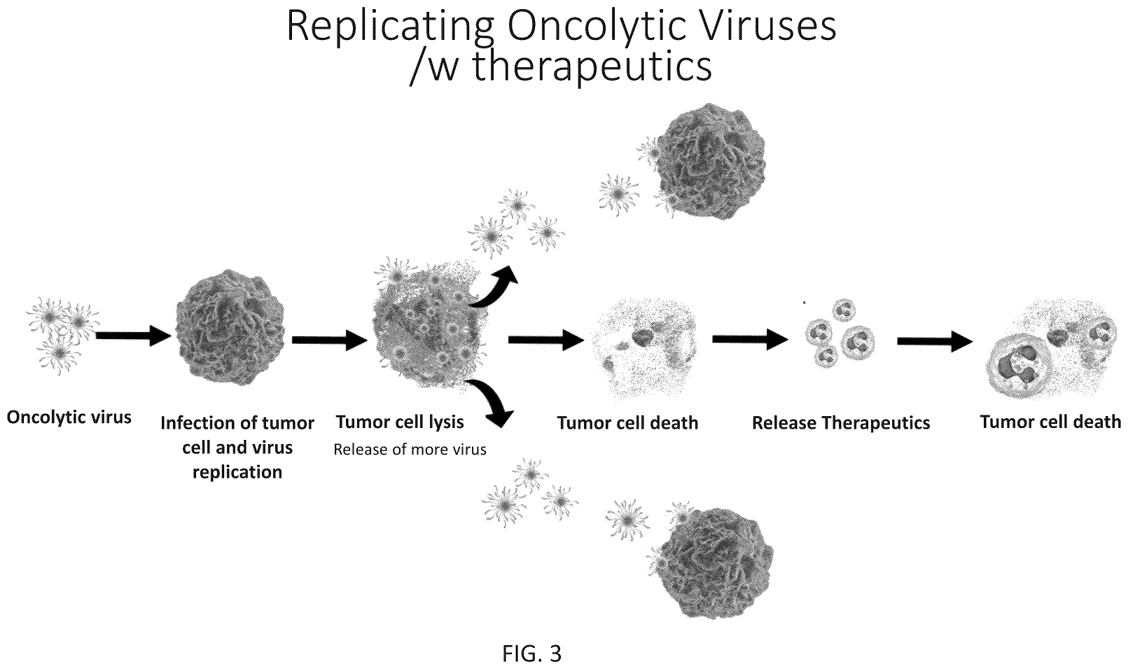

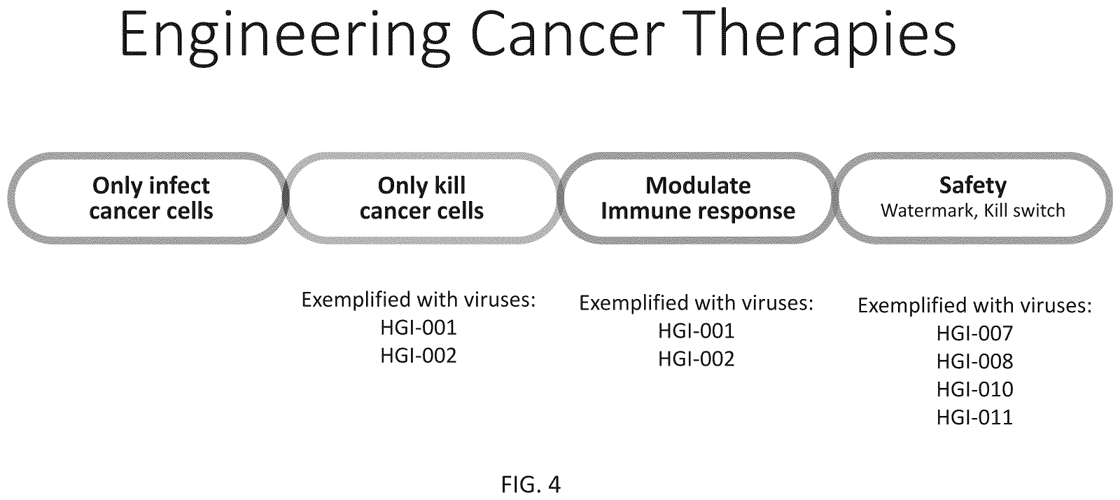

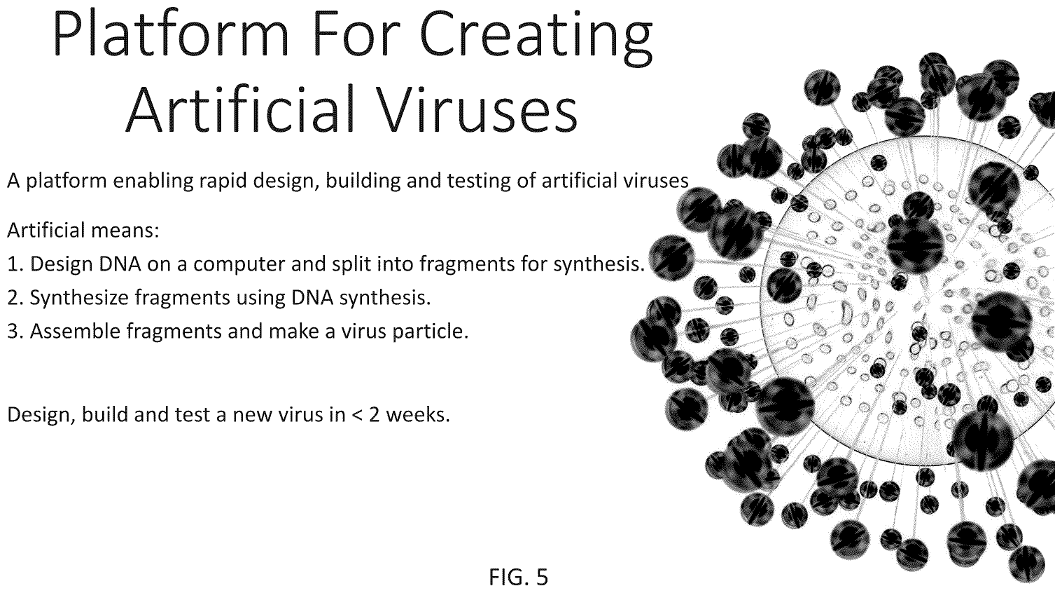

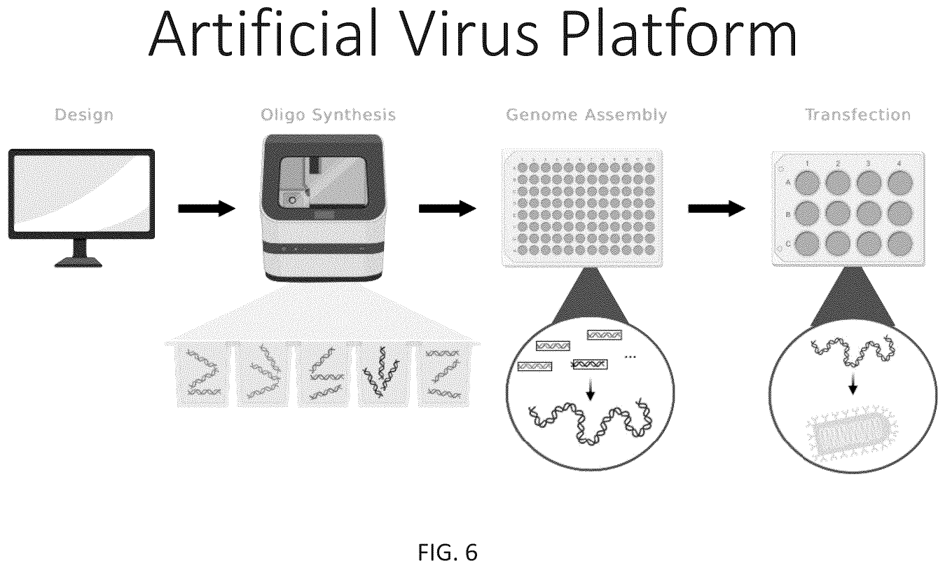

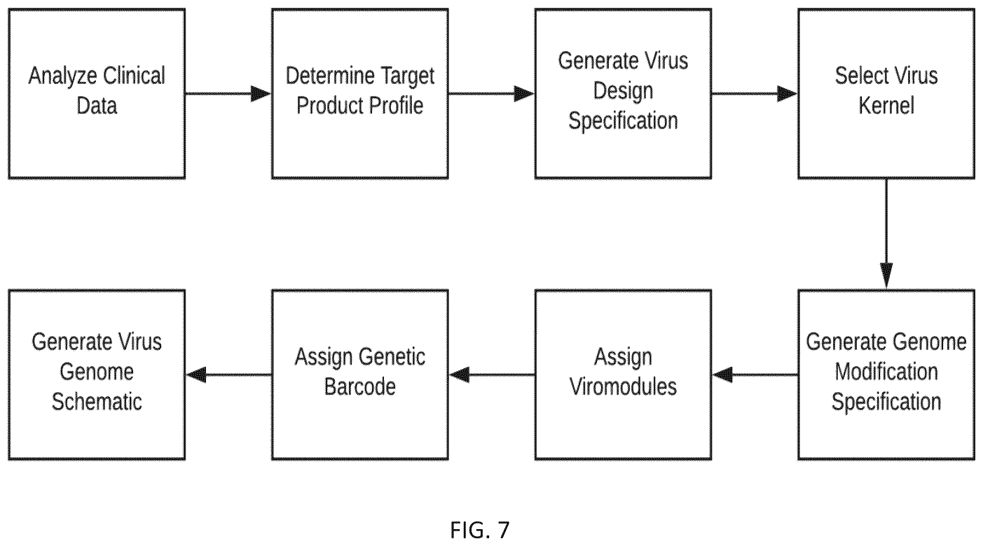

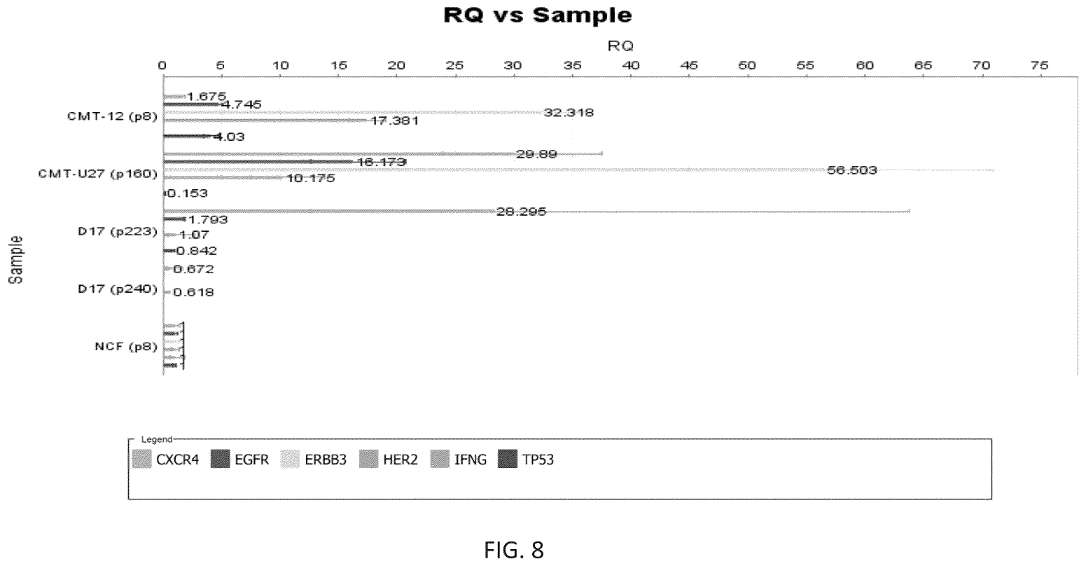

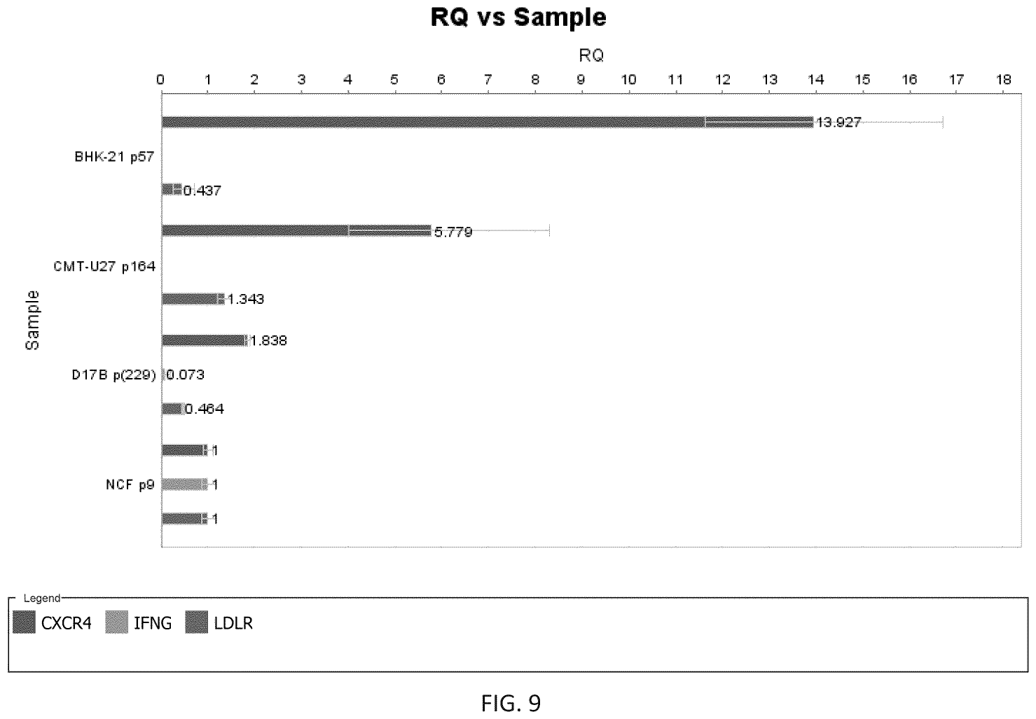

### DETAILED DESCRIPTION OF THE INVENTION

Oncolytic viruses are a subset of lytic viruses that selectively replicate in and lyse cancer cells with little or no effect on normal cells. Oncolytic viral therapies harness the basic biological principles of the virus; the virus replicates in cancer cells, and as the infected cancer cells are destroyed by oncolysis, they release new infectious virus particles or virions to help destroy the remaining cancer cells or tumor. Often these viruses replicate in dividing cells preferentially over non-dividing cells. Importantly, the viral replication cycle allows local amplification of the virus, and the oncolytic process continues as long as target cells exist. While the potential of oncolytic viral therapies has been recognized for some time, real world obstacles to broad utility of this therapeutic approach remain and are addressed by the inventions described herein. Although a degree of natural tumor-selectivity can be demonstrated for some virus species, new approaches are still needed to engineer and/or enhance tumor-selectivity for oncolytic viruses in order to maximize safety. This selectivity is particularly important when intravenous administration is used, and/or when potentially toxic therapeutic genes are added to the viruses to enhance antitumoral potency; gene expression must be tightly limited in normal tissues.

The inventions disclosed herein provide viral compositions for use in inducing the regression of a tumor or neoplasia, reducing the size of or eliminating a tumor or neoplasia, and treating or eliminating cancer (e.g., solid tumor cancers or blood cancers) or a hyperproliferative disorder in a subject to whom the compositions are administered. The viral compositions of the invention are artificial (i.e., non-naturally occurring and not identical to naturally occurring compositions), and in some embodiments fully synthetic (i.e., synthesized completely de novo as opposed to beginning with and modifying a naturally occurring virus). The inventions also provide methods of making these compositions and methods of using these compositions therapeutically, particularly methods of selectively killing cancer cells by contacting them with the viral compositions described herein.

Although numerous benefits can be derived from the inventions described herein, of particular note are the cancer cell targeting specificity (i.e., selective infection) and cancer cell replication specificity (i.e., selective replication) exhibited by the artificial viruses of the invention. Both specificities are generally conferred by engineering the artificial virus based on bioinformatic analysis of the cancer cell to be targeted as described further herein. The bioinformatic analysis can be conducted on one or more samples from a single individual (i.e., giving rise to a personalized therapeutic approach) or on groups of samples (stratified, for example, by type of cancer, origin of cancer, stage of cancer, time course of cancer progression, time course of cancer treatment, ethnicity, gender, age, etc.) to inform a pan-generic therapeutic approach. The bioinformatic analysis is conducted to identify genes that are differentially expressed between the cells of the sample(s) and normal or non-target cells, and/or to determine expression levels of differentially expressed genes. As a result of this analysis, one or more artificial oncolytic viruses can be engineered to preferentially or specifically bind to (and thereby infect) a cancer cell by binding to a gene product identified as preferentially or specifically expressed on the cancer cell. In certain embodiments the gene product is highly expressed on the cancer cell in addition to being preferentially or specifically expressed. Additionally or alternatively, one or more artificial oncolytic viruses can be engineered such that transcription or replication of the viral genome is under the control of a regulatory region, such as a promoter/enhancer, of a gene identified as preferentially or specifically expressed by the cancer cell, resulting in cell- or tumor-specific or -preferential expression or replication of the viral genome. In certain embodiments the gene product is highly expressed by the cancer cell in addition to being preferentially or specifically expressed. In this manner binding and/or expression of the artificial oncolytic viruses can be engineered to greatly reduce or eliminate binding to/infection of and/or replication in non-target cells. These and other advantages of the disclosed inventions will be apparent from the description.

### Definitions

Unless defined otherwise, all technical and scientific terms used herein have the meaning commonly understood by a person skilled in the art. The following references provide one of skill with a general definition of many of the terms used herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

Standard techniques may be used for recombinant DNA, oligonucleotide synthesis, tissue culture and transformation, protein purification, etc. Enzymatic reactions and purification techniques may be performed according to the manufacturer's specifications or as commonly accomplished in the art or as described herein. The following procedures and techniques may be generally performed according to conventional methods well known in the art and as described in various general and more specific references that are cited and discussed throughout the specification. See, e.g., Sambrook et al., 2001, Molecular Cloning: A Laboratory Manuel, 3.sup.rd ed., Cold Spring Harbor Laboratory Press, Cold Spring Harbor, N.Y., which is incorporated herein by reference for any purpose. Unless specific definitions are provided, the nomenclature used in connection with, and the laboratory procedures and techniques of, analytic chemistry, organic chemistry, and medicinal and pharmaceutical chemistry described herein are those well-known and commonly used in the art. Standard techniques may be used for chemical synthesis, chemical analyses, pharmaceutical preparation, formulation, and delivery and treatment of patients.

Viral Compositions

Embodiments of the invention relate to artificial oncolytic viruses comprising a domain engineered to selectively bind to a target cell via a binding partner identified as highly expressed or preferentially expressed on the target cell as compared with a non-target cell. In some embodiments the binding partner is both highly expressed and preferentially expressed on the target cell as compared with a non-target cell. Embodiments of the invention also relate to artificial oncolytic viruses wherein expression of the viral genome is engineered to be under the control of a regulatory region of a gene identified as highly expressed or preferentially expressed by the target cell as compared with a non-target cell. In some embodiments the gene is both highly expressed and preferentially expressed by the target cell as compared with a non-target cell. In some embodiments either or both of the binding partner and the gene are specifically expressed by the target cell. In some embodiments the target cell is a cancer cell, such as a tumor cell. In certain embodiments the artificial oncolytic virus both comprises a domain engineered to selectively bind to a target cell via a binding partner identified as highly expressed or preferentially expressed on the target cell as compared with a non-target cell and wherein expression of the viral genome is engineered to be under the control of a regulatory region of a gene identified as highly expressed or preferentially expressed by the target cell as compared with a non-target cell.

Viral compositions of the present invention are preferably non-pathogenic in a subject to whom the virus is intended to be administered. Additionally, viral compositions of the invention are preferably those to which a subject to whom it is intended to be administered has reduced or no pre-existing immunity, as it is desirable to delay any immune response to the virus to enable the virus to infect and destroy target cells without interference from the subject's immune system.

Viral compositions of the present invention, while in some embodiments similar to a reference naturally-occurring or wild type virus, are artificial and do not exist in nature. Viral compositions of the invention may be substantially identical to (i.e., derived from) a reference naturally-occurring virus. As used herein, an oncolytic virus which is derived from a reference virus comprises a nucleic acid sequence or amino acid sequence which is possessed by the reference virus. In some embodiments an oncolytic virus which is “derived from” a reference virus comprises one or more genes possessed by the reference virus. In some embodiments an oncolytic virus which is derived from a reference virus encodes one or more proteins encoded by the reference virus. In some embodiments, an oncolytic virus which is derived from a reference virus may comprise nucleic acid sequence encoding one or more functional elements of the reference virus. A “functional element” may, e.g., be a transcriptional regulator (e.g., a promoter/enhancer), a regulator of post-transcriptional processing, a translational regulator, a regulator of post-transcriptional processing, a response element, a repeat sequence, or a viral protein. In some embodiments, an oncolytic virus which is derived from a reference virus may comprise one or more genes of, or proteins encoded by, the reference virus.

In some embodiments, the virus comprises at least one domain which is identical to a domain of a reference naturally-occurring virus. In some embodiments the virus is identical to a reference naturally-occurring virus except for the engineered domain and/or the engineered regulatory domain.

Reference naturally-occurring viruses can include, but are not limited to measles virus, rabies virus, Gibbon Ape Leukemia Virus, Sendai Virus, Seneca valley virus (SVV), an adenovirus (Ad), herpes simplex virus (HSV), vaccinia virus (VV), vesicular stomatitis virus (VSV); autonomous parvovirus, myxoma virus (MYXV), Newcastle disease virus (NDV), reovirus, retrovirus, influenza virus, Sindbis virus (SINV) or poxvirus, as examples. For example, in some embodiments the reference naturally-occurring virus can be a member of the Rhabdoviridae family, such as from the genus Vesiculovirus. In certain instances, the reference naturally-occurring virus can be Indiana vesiculovirus or New Jersey vesiculovirus.

In some embodiments of the invention the artificial oncolytic virus additionally comprises one or more heterologous functional domains, wherein said functional domain is selected from the group consisting of a therapeutic agent, a kill switch for said target cell, an agent which facilitates the ability of the virus to evade the recipient immune system, a watermark, a barcode, an agent which degrades the extracellular matrix of a solid tumor, and a diagnostic agent.

In some embodiments, an oncolytic virus according to the present disclosure may possess one or more of the following functional properties: ability to replicate in, and/or cause cell killing of, cancer cells; reduced ability to replicate in and/or cause cell killing of, non-cancerous cells as compared to the ability to replicate in, and/or cause cell killing of, cancer cells; comparable or improved ability to cause cell killing of cancer cells as compared to the ability of one or more oncolytic viruses known in the art; ability to help replication of helper-dependent adenovirus (HDAd); comparable or improved ability to replicate in cancer cells as compared to the ability of one or more oncolytic viruses known in the art.

As used herein “wild-type” refers to the naturally occurring sequence of a nucleic acid at a genetic locus in the genome of an organism, and sequences transcribed or translated from such a nucleic acid. Thus, the term “wild-type” also may refer to the amino acid sequence encoded by the nucleic acid. As a genetic locus may have more than one sequence or alleles in a population of individuals, the term “wild-type” encompasses all such naturally occurring alleles. As used herein the term “polymorphic” means that variation exists (i.e., two or more alleles exist) at a genetic locus in the individuals of a population. As used herein, “mutant” refers to a change in the sequence of a nucleic acid or its encoded protein, polypeptide, or peptide that is the result of recombinant DNA technology.

A nucleic acid may be made by any technique known to one of ordinary skill in the art. Non-limiting examples of a synthetic nucleic acid, particularly a synthetic oligonucleotide, include a nucleic acid made by in vitro chemical synthesis using phosphotriester, phosphite or phosphoramidite chemistry and solid phase techniques such as described in EP 266,032, or via deoxynucleoside H-phosphonate intermediates as described by Froehler et al., 1986, and U.S. Pat. No. 5,705,629. A non-limiting example of enzymatically produced nucleic acid includes one produced by enzymes in amplification reactions such as PCR.TM. (see for example, U.S. Pat. Nos. 4,683,202 and 4,682,195), or the synthesis of oligonucleotides described in U.S. Pat. No. 5,645,897. A non-limiting example of a biologically produced nucleic acid includes recombinant nucleic acid production in living cells, such as recombinant DNA vector production in bacteria (see for example, Sambrook et al. 1989).

The nucleic acid(s), regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). The overall length may vary considerably between nucleic acid constructs. Thus, a nucleic acid segment of almost any length may be employed, with the total length preferably being limited by the ease of preparation or use in the intended recombinant nucleic acid protocol.

By “expression construct” or “expression cassette” is meant a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide to be delivered to a host cell, either in vitro or in vivo. A “plasmid,” a common type of a vector, is an extra-chromosomal DNA molecule separate from the chromosomal DNA that is capable of replicating independently of the chromosomal DNA. In certain cases, it is circular and double-stranded.

The term “promoter” is used herein in its ordinary sense to refer to a nucleotide region comprising a DNA regulatory sequence, wherein the regulatory sequence is derived from a gene that is capable of binding RNA polymerase and initiating transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

By “operably linked” or co-expressed” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” or “co-expressed” with reference to peptide and/or polypeptide molecules means that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of heterologous molecules.

Pharmaceutical Compositions and Methods of Treatment

Some embodiments of the present invention relate to methods of treatment for a hyperproliferative disease, such as cancer, by the delivery of a pharmaceutical composition comprising an effective amount of one or more artificial oncolytic viruses described herein. An effective amount of the pharmaceutical composition is an amount sufficient to induce oncolysis in a cell to which the composition is administered and/or the slowing, inhibition or reduction (including complete eradication) in the growth or size of a tumor and/or to treat a cancer in a subject to whom the composition is administered. The cytotoxic effects under in vitro or in vivo conditions can be detected by various means as known in the art, for example, by detecting tumor size using gadolinium enhanced MRI scanning, by radiolabeling of a tumor, and the like.

As used herein, the terms “treat” and “treating” refers to a treatment/therapy from which a subject receives a beneficial effect, such as the reduction, decrease, attenuation, diminishment, stabilization, remission, suppression, inhibition or arrest of the development or progression of cancer, or a symptom thereof. In certain embodiments, the treatment/therapy that a subject receives results in at least one or more of the following effects: (i) the reduction or amelioration of the severity of cancer and/or a symptom associated therewith; (ii) the reduction in the duration of a symptom associated with cancer; (iii) the prevention in the recurrence of a symptom associated with cancer; (iv) the regression of cancer and/or a symptom associated therewith; (v) the reduction in hospitalization of a subject; (vi) the reduction in hospitalization length; (vii) the increase in the survival of a subject; (viii) the inhibition of the progression of cancer and/or a symptom associated therewith; (ix) the enhancement of or improvement in the therapeutic effect of another therapy; (x) a reduction or elimination in the cancer cell population; (xi) a reduction in the growth of a tumor or neoplasm; (xii) a decrease in tumor size; (xiii) a reduction in the formation of a tumor; (xiv) eradication, removal, or control of primary, regional and/or metastatic cancer; (xv) a decrease in the number or size of metastases; (xvi) a reduction in mortality; (xvii) an increase in cancer-free survival rate of patients; (xviii) an increase in relapse-free survival; (xix) an increase in the number of patients in remission; (xx) a decrease in hospitalization rate; (xxi) the size of the tumor is maintained and does not increase in size or increases the size of the tumor by less than 5% or 10% after administration of a therapy as measured by conventional methods available to one of skill in the art, such as MRI, X-ray, and CAT Scan; (xxii) the prevention of the development or onset of cancer and/or a symptom associated therewith; (xxiii) an increase in the length of remission in a subject to whom the therapy is administered; (xxiv) the reduction in the number of symptoms associated with cancer; (xxv) an increase in symptom-free survival of cancer patients; and/or (xxvi) limitation of or reduction in metastasis. In some embodiments, the treatment/therapy that a subject receives does not cure cancer, but prevents the progression or worsening of the disease. In certain embodiments, the treatment/therapy that a subject receives does not prevent the onset/development of cancer, but may prevent the onset of cancer symptoms.

As used herein, the terms “patient” or “subject” are used interchangeably and mean a mammal, including, but not limited to, a human or non-human mammal, such as a bovine, equine, canine, ovine, or feline. Preferably, the patient is a human (Homo sapiens) or a canine (e.g., Canis lupus familiaris). The subject may be of any gender. A subject may have been diagnosed with a cancer requiring treatment, may be suspected of having such a cancer, or may be at risk of developing such a cancer.

Examples of cancer contemplated for treatment in accordance with the invention include, but are not limited to, liver cancer, lung cancer, head and neck cancer, breast cancer, pancreatic cancer, prostate cancer, renal cancer, bone cancer, testicular cancer, cervical cancer, gastrointestinal cancer, leukemias, lymphomas, pre-neoplastic lesions in the lung, colon cancer, melanoma, and bladder cancer.

In some embodiments the composition is administered to a subject who has a tumor. The tumor can be, for example, a brain cancer tumor, a head & neck cancer tumor, an esophageal cancer tumor, a skin cancer tumor, a lung cancer tumor, a thymic cancer tumor, a stomach cancer tumor, a colon cancer tumor, a liver cancer tumor, an ovarian cancer tumor, a uterine cancer tumor, a bladder cancer tumor, a testicular cancer tumor, a rectal cancer tumor, a breast cancer tumor, or a pancreatic cancer tumor. The tumor can be a primary tumor or a metastatic tumor or a recurrent tumor.

Cancer cells that may be treated by methods and compositions of the invention include cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

The present invention contemplates methods for inhibiting or preventing local invasiveness and/or metastasis of any type of primary cancer. For example, the primary cancer may be melanoma, non-small cell lung, small-cell lung, lung, hepatocarcinoma, retinoblastoma, astrocytoma, glioblastoma, gum, tongue, leukemia, neuroblastoma, head, neck, breast, lung, pancreatic, prostate, renal, bone, testicular, ovarian, mesothelioma, cervical, gastrointestinal, lymphoma, brain, colon, or bladder. Moreover, the present invention can be used to prevent cancer or to treat pre-cancers or premalignant cells, including metaplasias, dysplasias, and hyperplasias. It may also be used to inhibit undesirable but benign cells, such as squamous metaplasia, dysplasia, benign prostate hyperplasia cells, hyperplastic lesions, and the like. The progression to cancer or to a more severe form of cancer may be halted, disrupted, or delayed by methods of the invention as discussed herein.

Administration

In accordance with methods of the invention, treatment comprises contacting one or more cancer cells or tumors with a composition according to the invention. The routes of administration will vary, naturally, with the location and nature of the lesion, and include, e.g., intradermal, transdermal, parenteral, intravenous, intramuscular, intranasal, subcutaneous, regional (e.g., in the proximity of a tumor, particularly with the vasculature or adjacent vasculature of a tumor), percutaneous, intratracheal, intraperitoneal, intraarterial, intravesical, intratumoral, inhalation, perfusion, lavage, and oral administration and formulation. Treatment regimens may vary as well, and often depend on tumor type, tumor location, disease progression, and health and age of the patient.

The term “intravascular” is understood to refer to delivery into the vasculature of a patient, meaning into, within, or in a vessel or vessels of the patient. In certain embodiments, the administration is into a vessel considered to be a vein (intravenous), while in others administration is into a vessel considered to be an artery. Veins include, but are not limited to, the internal jugular vein, a peripheral vein, a coronary vein, a hepatic vein, the portal vein, great saphenous vein, the pulmonary vein, superior vena cava, inferior vena cava, a gastric vein, a splenic vein, inferior mesenteric vein, superior mesenteric vein, cephalic vein, and/or femoral vein. Arteries include, but are not limited to, coronary artery, pulmonary artery, brachial artery, internal carotid artery, aortic arch, femoral artery, peripheral artery, and/or ciliary artery. It is contemplated that delivery may be through or to an arteriole or capillary.

Intratumoral injection, or injection directly into the tumor vasculature, is specifically contemplated for discrete, solid, accessible tumors. Local, regional or systemic administration also may be appropriate. In some embodiments the volume to be administered can be, for example, up to 5 mL at a concentration of 10⁹ plaque-forming units (PFU) or fluorescent-forming units (FFU) per mL. The viral particles may advantageously be delivered by administering multiple injections to the tumor, spaced at approximately 1 cm intervals.

Continuous administration also may be applied where appropriate, for example, by implanting a catheter into a tumor or into tumor vasculature. Such continuous perfusion may take place for a period from about 1-2 hours, to about 2-6 hours, to about 6-12 hours, or about 12-24 hours following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

In the case of surgical intervention, the present invention may be used preoperatively, to render an inoperable tumor suitable for resection or to address non-resected cells that may remain in the subject locally or metastatically. In certain embodiments, the tumor being treated may not, at least initially, be resectable. Treatments with therapeutic viral compositions may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

The treatments may include various “unit doses” defined as containing a predetermined-quantity of the therapeutic composition. The quantity to be administered, and the particular route and formulation, are within the skill of those in the clinical arts. A unit dose need not be administered as a single injection but may comprise continuous infusion over a specified period of time. Unit dose of the present invention may conveniently be described in terms of plaque forming units (pfu) for a viral construct.

The pharmaceutical compositions disclosed herein may be administered intratumorally, parenterally, intravenously, intradermally, intramuscularly, transdermally or even intraperitoneally as described in U.S. Pat. Nos. 5,543,158; 5,641,515 and 5,399,363.

Injection of nucleic acid constructs may be delivered by syringe or any other method used for injection of a solution, as long as the expression construct can pass through the particular gauge of needle required for injection and the dosage can be administered with the required level of precision.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The phrase “pharmaceutically-acceptable” or “pharmacologically-acceptable” refers to molecular entities and compositions that do not produce an allergic or similar untoward reaction when administered to a subject (e.g., a canine or a human). As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Except insofar as any conventional media or agent is incompatible with the viral agent, its use in the therapeutic compositions is contemplated. Supplementary active ingredients can also be incorporated into the compositions.

Combination Therapies

The compositions and methods of the present invention may be used in the context of hyperproliferative diseases/conditions including cancer. In order to increase the effectiveness of a treatment with the compositions of the present invention, it may be desirable to combine these compositions with other agents effective in the treatment of those diseases and conditions. For example, the treatment of a cancer may be implemented with therapeutic compositions of the present invention in combination with other anti-cancer therapies, such as anti-cancer agents or surgery. As used herein, the term “in combination” in the context of the administration of (a) therapy(ies) to a subject, refers to the use of more than one therapy. The use of the term “in combination” does not restrict the order in which therapies are administered to a subject. A first therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

Administration of the viral compositions of the present invention to a patient in combination with a secondary therapy will follow general protocols for the administration of that particular secondary therapy, repeating treatment cycles as necessary. The secondary anti-cancer agent or therapy can be one or more therapies selected from the group consisting of, for example, chemotherapy, biological therapy, radiotherapy, immunotherapy, hormone therapy, anti-vascular therapy, cryotherapy, toxin therapy and surgery.

As used herein, an anti-cancer agent or therapy is capable of negatively affecting cancer in a subject, for example, by killing cancer cells, inducing apoptosis, necrosis, and/or cytopathic effect (CPE) in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. More generally, these other compositions would be provided in a combined amount effective to kill or inhibit proliferation of the cell. This process may involve contacting the cells with the viral composition and the secondary therapy or agent at the same time. This may be achieved by contacting the cell with a single composition or pharmacological formulation that includes both agents, or by contacting the cell with two distinct compositions or formulations, at the same time, wherein one composition includes the viral expression construct and the other includes the second agent(s).

Alternatively, the viral therapy may precede or follow the other agent treatment by intervals ranging from minutes to weeks. In embodiments where the other agent and artificial virus are applied separately to the cell, one would generally ensure that a significant period of time did not expire between the time of each delivery, such that the agent and virus would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

The inventions disclosed herein will be exemplified in a non-limiting manner by the following. The teachings of all references, patents and patent applications cited herein are incorporated fully by reference herein.

### Examples

Methods

In Silico Analysis of Cancer Datasets

Online repositories and publicly available databases containing next-generation sequencing data for cancer cases were accessed to identify cancer-specific biomarkers and determine alteration frequencies of genes (e.g., amplification, deletion, mutation, fusion, multiple). These include The International Cancer Genome Consortium (ICGC), The Cancer Genome Atlas (TCGA), and Pan-Cancer Analysis of Whole Genomes (PCAWG). ANGPTL2, COL3A1, COL5A3, CXCR4, EGFR, ERBB2, ERBB3, ERBB4, FGFR1, FGFR2, FGFR3, FGFR4, HAS1, HIF1A, MET, MKI67, KIT, IFG1R, JAK2, PDGFRA, SPARC were identified as targets of interest. External references were used for further characterization and supplemental information, including NCBI, UniProt, Cancer Cell Line Encyclopedia, and cBioPortal).

Cell Culture

BHK-21, CMT-U27, and D17 cell lines were purchased from ATCC. CnOb were purchased from Cell Applications. CF11, D22, DKCre, FDK, HEK293, and NCF were a kind gift of Dr. Bruce Smith (Auburn University). CMT-12 and CMT-28 were a kind gift of Dr. Curtis Bird (Auburn University). Additional cell lines of interest were also objected, including OSCA-8, OSCA-32, OSCA-78, U2-OS, and HOS. Cells were maintained in DMEM (Corning) supplemented with 10% FBS (Gibco), 5% Penicillin/Streptomycin (Corning), and 1% Amphotericin B (Corning) at 37C and 5% CO2, with the exception that CnOb was cultured in osteoblast basal medium with growth supplement (Cell Applications).

Molecular Characterization

Cell lines were cultured under standard conditions and harvested for downstream PCR applications. Total RNA was isolated (from cell lysate) using TRIzol Reagent (Invitrogen), then reverse transcription was conducted to generate complementary DNA (cDNA) from the RNA templates using the High Capacity RNA-to-cDNA Kit (ThermoFisher). Alternatively, Cells-to-cDNA Kits were used for rapid cell lysis, RNA isolation, and cDNA synthesis. To determine expression of cancer biomarkers and targets, TaqMan Gene Expression Assays (Applied Biosystems) were used according to the manufacturer's protocols. Targets include ANGPTL2, B-actin, CASP3, CASP8, COL3A1, COL5A3, CXCR4, ERBB1/EGFR, ERBB2/HER2, ERBB3/HER3, ERBB4/HER4, FGFR1, GAPDH, HAS1, HAS2, HIF1A, IFNG, IGF1R, IL-6, IL-10, LDLR, MKI67, MUC1, MYC, OMD, OSM, SPARC, TEM8, TERT, TP53, VEGFA, among others. Real-time PCR was conducted using the QuantStudio 6 Flex (Applied Biosystems). Relative C t values were determined by comparison to control genes (e.g. GAPDH) and cell lines (e.g. NCF).

Next-generation sequencing and transcriptome analysis of cancer samples and cell lines was conducted to identify commonly over-expressed genes in cancer, tumor-specific biomarkers, and over-expressed proteins in cancer. For RNA-sequencing, tumor and healthy tissue samples were collected, purchased, or received from collaborators, high quality RNA samples were used for library preparation, and pooled libraries were sequenced with Illumina's NGS platform. Reads were aligned to canine reference genome (CanFam3.1). Count tables were used for analysis and determining differential expression. Enrichment analysis of bulk data was performed using Gene Set Enrichment Analysis (GSEA). Fold change for transcripts were calculated using normalized gene expression of tumor samples and normal samples. Variants calling was performed and filtered for differential expression in tumor samples.

Virus Genome Engineering

To develop oncolytic virus genomes with cancer-specific targeting and replication, a collection of biodesign algorithms and computer-aided design software was used. Source genetic material (e.g., single gene, whole genome) from a wildtype virus was downloaded from a public database (e.g., NCBI Genbank) as a FASTA file.

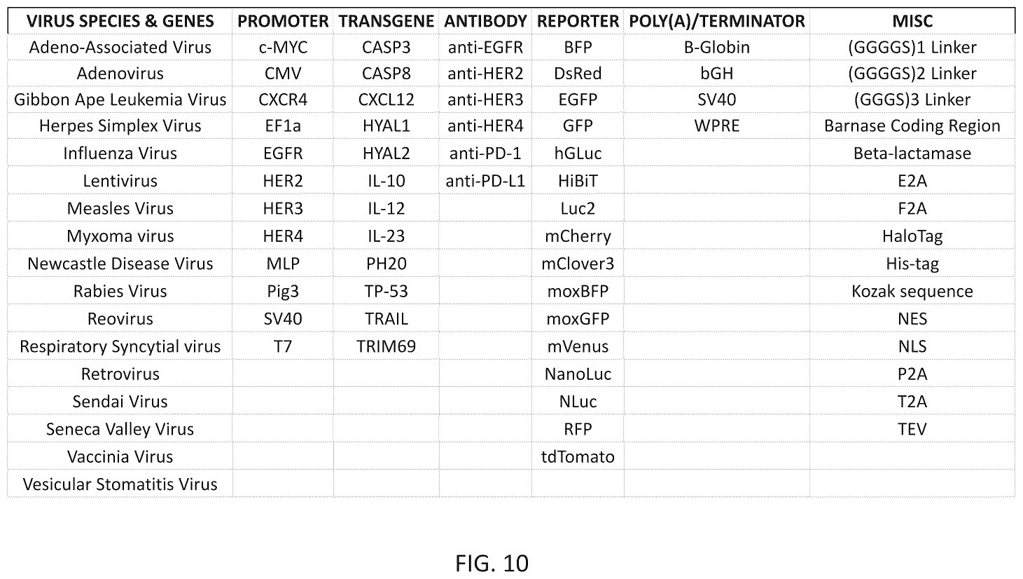

The core components that may be utilized in the design and manufacture of an oncolytic virus described herein are provided in FIG. 10. Examples of different virus species and genes, promoters, transgenes, antibodies, reporters, poly(a)/terminators, and other miscellaneous components that may be used in combination to form an oncolytic virus as disclosed herein are provided. Individual components from each category may be chosen for the design and manufacture of a specific oncolytic virus. The selection of specific components may be influenced by their size, such that certain combinations of components may be undesirable, i.e., the components would be too large in combination for delivery in an oncolytic virus.

An example of a modularized vesicular stomatitis virus (Indiana Strain), (Reference: HGI-007.1) is outlined here:

| | |
|---|---|
| mRNA | 51..1376 |
| | /product= “N mRNA” |
| | /note= “Nucleocapsid” |
| mRNA | 1386..2199 |
| | /product= “P mRNA” |
| | /note= “Phosphoprotein” |
| mRNA | 2209..3039 |
| | /product= “M mRNA” |
| | /note= “Matrix” |
| Variation | 3047..3094 |
| | /product= “VA1” |
| | /note= “Viral Adaptor 1” |
| Variation | 3095..3136 |
| | /product= “VA2” |
| | /note= “Viral Adaptor 2” |
| mRNA | 3139..4803 |
| | /product= “G mRNA” |
| | /note= “Glycoprotein” |
| Variation | 4811..4855 |
| | /product= “VA3” |
| | /note= “Viral Adaptor 3” |
| Variation | 4856..4912 |
| | /product= “VA4” |
| | /note= “Viral Adaptor 4” |
| mRNA | 4915..11287 |
| | /product= “L mRNA” |
| | /note= “Polymerase” |
| ORIGIN | |

(SEQ ID NO: 2)
ACGAAGACAAACAAACCATTATTATCATTAAAAGGCTCAG
GAGAAACTTTAACAGTAATCAAAATGTCTGTTACAGTCAA
GAGAATCATTGACAACACAGTCATAGTTCCAAAACTTCCT
GCAAATGAGGATCCAGTGGAATACCCGGCAGATTACTTCA
GAAAATCAAAGGAGATTCCTCTTTACATCAATACTACAAA
AAGTTTGTCAGATCTAAGAGGATATGTCTACCAAGGCCTC
AAATCCGGAAATGTATCAATCATACATGTCAACAGCTACT
TGTATGGAGCATTAAAGGACATCCGGGGTAAGTTGGATAA
AGATTGGTCAAGTTTCGGAATAAACATCGGGAAAGCAGGG
GATACAATCGGAATATTTGACCTTGTATCCTTGAAAGCCC
TGGACGGCGTACTTCCAGATGGAGTATCGGATGCTTCCAG
AACCAGCGCAGATGACAAATGGTTGCCTTTGTATCTACTT
GGCTTATACAGAGTGGGCAGAACACAAATGCCTGAATACA
GAAAAAAGCTCATGGATGGGCTGACAAATCAATGCAAAAT
GATCAATGAACAGTTTGAACCTCTTGTGCCAGAAGGTCGT
GACATTTTTGATGTGTGGGGAAATGACAGTAATTACACAA
AAATTGTCGCTGCAGTGGACATGTTCTTCCACATGTTCAA
AAAACATGAATGTGCCTCGTTCAGATACGGAACTATTGTT
TCCAGATTCAAAGATTGTGCTGCATTGGCAACATTTGGAC
ACCTCTGCAAAATAACCGGAATGTCTACAGAAGATGTAAC
GACCTGGATCTTGAACCGAGAAGTTGCAGATGAAATGGTC
CAAATGATGCTTCCAGGCCAAGAAATTGACAAGGCCGATT
CATACATGCCTTATTTGATCGACTTTGGATTGTCTTCTAA
GTCTCCATATTCTTCCGTCAAAAACCCTGCCTTCCACTTC
TGGGGGCAATTGACAGCTCTTCTGCTCAGATCCACCAGAG
CAAGGAATGCCCGACAGCCTGATGACATTGAGTATACATC
TCTTACTACAGCAGGTTTGTTGTACGCTTATGCAGTAGGA
TCCTCTGCCGACTTGGCACAACAGTTTTGTGTTGGAGATA
ACAAATACACTCCAGATGATAGTACCGGAGGATTGACGAC
TAATGCACCGCCACAAGGCAGAGATGTGGTCGAATGGCTC
GGATGGTTTGAAGATCAAAACAGAAAACCGACTCCTGATA
TGATGCAGTATGCGAAAAGAGCAGTCATGTCACTGCAAGG
CCTAAGAGAGAAGACAATTGGCAAGTATGCTAAGTCAGAA
TTTGACAAATGACCCTATAATTCTCAGATCACCTATTATA
TATTATGCTACATATGAAAAAAACTAACAGATATCATGGA
TAATCTCACAAAAGTTCGTGAGTATCTCAAGTCCTATTCT
CGTCTGGATCAGGCGGTAGGAGAGATAGATGAGATCGAAG
CACAACGAGCTGAAAAGTCCAATTATGAGTTGTTCCAAGA
GGATGGAGTGGAAGAGCATACTAAGCCCTCTTATTTTCAG
GCAGCAGATGATTCTGACACAGAATCTGAACCAGAAATTG
AAGACAATCAAGGTTTGTATGCACAGGATCCAGAAGCTGA
GCAAGTTGAAGGCTTTATACAGGGGCCTTTAGATGACTAT
GCAGATGAGGAAGTGGATGTTGTATTTACTTCGGACTGGA
AACCACCTGAGCTTGAATCTGACGAGCATGGAAAGACCTT
ACGGTTGACATCGCCAGAGGGTTTAAGTGGAGAGCAGAAA
TCCCAGTGGCTTTCGACGATTAAAGCAGTCGTGCAAAGTG
CCAAATACTGGAATCTGGCAGAGTGCACATTTGAAGCATC
GGGAGAAGGGGTCATTATGAAGGAGCGCCAGATAACTCCG
GATGTATATAAGGTCACTCCAGTGATGAACACACATCCGT
CCCAATCAGAAGCAGTATCAGATGTTTGGTCTCTCTCAAA
GACATCCATGACTTTCCAACCCAAGAAAGCAAGTCTTCAG
CCTCTCACCATATCCTTGGATGAATTGTTCTCATCTAGAG
GAGAGTTCATCTCTGTCGGAGGTGACGGACGAATGTCTCA
TAAAGAGGCCATCCTGCTCGGCCTGAGATACAAAAAGTTG
TACAATCAGGCGAGAGTCAAATATTCTCTGTAGACTATGA
AAAAAAGTAACAGATATCACGATCTAAGTGTTATCCCAAT
CCATTCATCATGAGTTCCTTAAAGAAGATTCTCGGTCTGA
AGGGGAAAGGTAAGAAATCTAAGAAATTAGGGATCGCACC
ACCCCCTTATGAAGAGGACACTAGCATGGAGTATGCTCCG
AGCGCTCCAATTGACAAATCCTATTTTGGAGTTGACGAGA
TGGACACCTATGATCCGAATCAATTAAGATATGAGAAATT
CTTCTTTACAGTGAAAATGACGGTTAGATCTAATCGTCCG
TTCAGAACATACTCAGATGTGGCAGCCGCTGTATCCCATT
GGGATCACATGTACATCGGAATGGCAGGGAAACGTCCCTT
CTACAAAATCTTGGCTTTTTTGGGTTCTTCTAATCTAAAG
GCCACTCCAGCGGTATTGGCAGATCAAGGTCAACCAGAGT
ATCACACTCACTGCGAAGGCAGGGCTTATTTGCCACATAG
GATGGGGAAGACCCCTCCCATGCTCAATGTACCAGAGCAC
TTCAGAAGACCATTCAATATAGGTCTTTACAAGGGAACGA
TTGAGCTCACAATGACCATCTACGATGATGAGTCACTGGA
AGCAGCTCCTATGATCTGGGATCATTTCAATTCTTCCAAA
TTTTCTGATTTCAGAGAGAAGGCCTTAATGTTTGGCCTGA
TTGTCGAGAAAAAGGCATCTGGAGCGTGGGTCCTGGATTC
TATCAGCCACTTCAAATGAGCTAGTCTAACTTCTAGCTTC
TGAACAATCCCCGGTTTACTCAGTCTCTCCTAATTCCAGC
CTCTCGAACAACTAATATCCTGTCTTTTCTATCCCTATGA
AAAAAATTTTCATAGATTCAACTGTTTTCATAGTAAAACC
AACGTAACTAAGCTTCATCCCAATAGTGCTAATACTAATG
CCGTCAACTGTTTGCTCTAACAGAGATCGATCTGTTTCCT
TGACACTATGAAGTGCCTTTTGTACTTAGCCTTTTTATTC
ATTGGGGTGAATTGCAAGTTCACCATAGTTTTTCCACACA
ACCAAAAAGGAAACTGGAAAAATGTTCCTTCTAATTACCA
TTATTGCCCGTCAAGCTCAGATTTAAATTGGCATAATGAC
TTAATAGGCACAGCCATACAAGTCAAAATGCCCAAGAGTC
ACAAGGCTATTCAAGCAGACGGTTGGATGTGTCATGCTTC
CAAATGGGTCACTACTTGTGATTTCCGCTGGTATGGACCG
AAGTATATAACACAGTCCATCCGATCCTTCACTCCATCTG
TAGAACAATGCAAGGAAAGCATTGAACAAACGAAACAAGG
AACTTGGCTGAATCCAGGCTTCCCTCCTCAAAGTTGTGGA
TATGCAACTGTGACGGATGCCGAAGCAGTGATTGTCCAGG
TGACTCCTCACCATGTGCTGGTTGATGAATACACAGGAGA
ATGGGTTGATTCACAGTTCATCAACGGAAAATGCAGCAAT
TACATATGCCCCACTGTCCATAACTCTACAACCTGGCATT
CTGACTATAAGGTCAAAGGGCTATGTGATTCTAACCTCAT
TTCCATGGACATCACCTTCTTCTCAGAGGACGGAGAGCTA
TCATCCCTGGGAAAGGAGGGCACAGGGTTCAGAAGTAACT
ACTTTGCTTATGAAACTGGAGGCAAGGCCTGCAAAATGCA
ATACTGCAAGCATTGGGGAGTCAGACTCCCATCAGGTGTC
TGGTTCGAGATGGCTGATAAGGATCTCTTTGCTGCAGCCA
GATTCCCTGAATGCCCAGAAGGGTCAAGTATCTCTGCTCC
ATCTCAGACCTCAGTGGATGTAAGTCTAATTCAGGACGTT
GAGAGGATCTTGGATTATTCCCTCTGCCAAGAAACCTGGA
GCAAAATCAGAGCGGGTCTTCCAATCTCTCCAGTGGATCT
CAGCTATCTTGCTCCTAAAAACCCAGGAACCGGTCCTGCT
TTCACCATAATCAATGGTACCCTAAAATACTTTGAGACCA
GATACATCAGAGTCGATATTGCTGCTCCAATCCTCTCAAG
AATGGTCGGAATGATCAGTGGAACTACCACAGAAAGGGAA
CTGTGGGATGACTGGGCACCATATGAAGACGTGGAAATTG
GACCCAATGGAGTTCTGAGGACCAGTTCAGGATATAAGTT
TCCTTTATACATGATTGGACATGGTATGTTGGACTCCGAT
CTTCATCTTAGCTCAAAGGCTCAGGTGTTCGAACATCCTC
ACATTCAAGACGCTGCTTCGCAACTTCCTGATGATGAGAG
TTTATTTTTTGGTGATACTGGGCTATCCAAAAATCCAATC
GAGCTTGTAGAAGGTTGGTTCAGTAGTTGGAAAAGCTCTA
TTGCCTCTTTTTTCTTTATCATAGGGTTAATCATTGGACT
ATTCTTGGTTCTCCGAGTTGGTATCCATCTTTGCATTAAA
TTAAAGCACACCAAGAAAAGACAGATTTATACAGACATAG
AGATGAACCGACTTGGAAAGTAACTCAAATCCTGCACAAC
AGATTCTTCATGTTTGGACCAAATCAACTTGTGATACCAT
GCTCAAAGAGGCCTCAATTATATTTGAGTTTTTAATTTTT
ATGAAAAAAATTTTCCCTATAAATAGGCCGTCTAATATTT
TAGTGCTTTTAACTAGCATAATAAACAATACAACGTTTGC
TGTGCTACATAGGCCGTCTAGTCTAGCTAATTCTAACAGC
AATCATGGAAGTCCACGATTTTGAGACCGACGAGTTCAAT
GATTTCAATGAAGATGACTATGCCACAAGAGAATTCCTGA
ATCCCGATGAGCGCATGACGTACTTGAATCATGCTGATTA
CAATTTGAATTCTCCTCTAATTAGTGATGATATTGACAAT
TTGATCAGGAAATTCAATTCTCTTCCGATTCCCTCGATGT
GGGATAGTAAGAACTGGGATGGAGTTCTTGAGATGTTAAC
ATCATGTCAAGCCAATCCCATCTCAACATCTCAGATGCAT
AAATGGATGGGAAGTTGGTTAATGTCTGATAATCATGATG
CCAGTCAAGGGTATAGTTTTTTACATGAAGTGGACAAAGA
GGCAGAAATAACATTTGACGTGGTGGAGACCTTCATCCGC
GGCTGGGGCAACAAACCAATTGAATACATCAAAAAGGAAA
GATGGACTGACTCATTCAAAATTCTCGCTTATTTGTGTCA
AAAGTTTTTGGACTTACACAAGTTGACATTAATCTTAAAT
GCTGTCTCTGAGGTGGAATTGCTCAACTTGGCGAGGACTT
TCAAAGGCAAAGTCAGAAGAAGTTCTCATGGAACGAACAT
ATGCAGGATTAGGGTTCCCAGCTTGGGTCCTACTTTTATT
TCAGAAGGATGGGCTTACTTCAAGAAACTTGATATTCTAA
TGGACCGAAACTTTCTGTTAATGGTCAAAGATGTGATTAT
AGGGAGGATGCAAACGGTGCTATCCATGGTATGTAGAATA
GACAACCTGTTCTCAGAGCAAGACATCTTCTCCCTTCTAA
ATATCTACAGAATTGGAGATAAAATTGTGGAGAGGCAGGG
AAATTTTTCTTATGACTTGATTAAAATGGTGGAACCGATA
TGCAACTTGAAGCTGATGAAATTAGCAAGAGAATCAAGGC
CTTTAGTCCCACAATTCCCTCATTTTGAAAATCATATCAA
GACTTCTGTTGATGAAGGGGCAAAAATTGACCGAGGTATA
AGATTCCTCCATGATCAGATAATGAGTGTGAAAACAGTGG
ATCTCACACTGGTGATTTATGGATCGTTCAGACATTGGGG
TCATCCTTTTATAGATTATTACACTGGACTAGAAAAATTA
CATTCCCAAGTAACCATGAAGAAAGATATTGATGTGTCAT
ATGCAAAAGCACTTGCAAGTGATTTAGCTCGGATTGTTCT
ATTTCAACAGTTCAATGATCATAAAAAGTGGTTCGTGAAT
GGAGACTTGCTCCCTCATGATCATCCCTTTAAAAGTCATG
TTAAAGAAAATACATGGCCCACAGCTGCTCAAGTTCAAGA
TTTTGGAGATAAATGGCATGAACTTCCGCTGATTAAATGT
TTTGAAATACCCGACTTACTAGACCCATCGATAATATACT
CTGACAAAAGTCATTCAATGAATAGGTCAGAGGTGTTGAA
ACATGTCCGAATGAATCCGAACACTCCTATCCCTAGTAAA
AAGGTGTTGCAGACTATGTTGGACACAAAGGCTACCAATT
GGAAAGAATTTCTTAAAGAGATTGATGAGAAGGGCTTAGA
TGATGATGATCTAATTATTGGTCTTAAAGGAAAGGAGAGG
GAACTGAAGTTGGCAGGTAGATTTTTCTCCCTAATGTCTT
GGAAATTGCGAGAATACTTTGTAATTACCGAATATTTGAT
AAAGACTCATTTCGTCCCTATGTTTAAAGGCCTGACAATG
GCGGACGATCTAACTGCAGTCATTAAAAAGATGTTAGATT
CCTCATCCGGCCAAGGATTGAAGTCATATGAGGCAATTTG
CATAGCCAATCACATTGATTACGAAAAATGGAATAACCAC
CAAAGGAAGTTATCAAACGGCCCAGTGTTCCGAGTTATGG
GCCAGTTCTTAGGTTATCCATCCTTAATCGAGAGAACTCA
TGAATTTTTTGAGAAAAGTCTTATATACTACAATGGAAGA
CCAGACTTGATGCGTGTTCACAACAACACACTGATCAATT
CAACCTCCCAACGAGTTTGTTGGCAAGGACAAGAGGGTGG
ACTGGAAGGTCTACGGCAAAAAGGATGGACTATCCTCAAT
CTACTGGTTATTCAAAGAGAGGCTAAAATCAGAAACACTG
CTGTCAAAGTCTTGGCACAAGGTGATAATCAAGTTATTTG
CACACAGTATAAAACGAAGAAATCGAGAAACGTTGTAGAA
TTACAGGGTGCTCTCAATCAAATGGTTTCTAATAATGAGA
AAATTATGACTGCAATCAAAATAGGGACAGGGAAGTTAGG
ACTTTTGATAAATGACGATGAGACTATGCAATCTGCAGAT
TACTTGAATTATGGAAAAATACCGATTTTCCGTGGAGTGA
TTAGAGGGTTAGAGACCAAGAGATGGTCACGAGTGACTTG
TGTCACCAATGACCAAATACCCACTTGTGCTAATATAATG
AGCTCAGTTTCCACAAATGCTCTCACCGTAGCTCATTTTG
CTGAGAACCCAATCAATGCCATGATACAGTACAATTATTT
TGGGACATTTGCTAGACTCTTGTTGATGATGCATGATCCT
GCTCTTCGTCAATCATTGTATGAAGTTCAAGATAAGATAC
CGGGCTTGCACAGTTCTACTTTCAAATACGCCATGTTGTA
TTTGGACCCTTCCATTGGAGGAGTGTCGGGCATGTCTTTG
TCCAGGTTTTTGATTAGAGCCTTCCCAGATCCCGTAACAG
AAAGTCTCTCATTCTGGAGATTCATCCATGTACATGCTCG
AAGTGAGCATCTGAAGGAGATGAGTGCAGTATTTGGAAAC
CCCGAGATAGCCAAGTTTCGAATAACTCACATAGACAAGC
TAGTAGAAGATCCAACCTCTCTGAACATCGCTATGGGAAT
GAGTCCAGCGAACTTGTTAAAGACTGAGGTTAAAAAATGC
TTAATCGAATCAAGACAAACCATCAGGAACCAGGTGATTA
AGGATGCAACCATATATTTGTATCATGAAGAGGATCGGCT
CAGAAGTTTCTTATGGTCAATAAATCCTCTGTTCCCTAGA
TTTTTAAGTGAATTCAAATCAGGCACTTTTTTGGGAGTCG
CAGACGGGCTCATCAGTCTATTTCAAAATTCTCGTACTAT
TCGGAACTCCTTTAAGAAAAAGTATCATAGGGAATTGGAT
GATTTGATTGTGAGGAGTGAGGTATCCTCTTTGACACATT
TAGGGAAACTTCATTTGAGAAGGGGATCATGTAAAATGTG
GACATGTTCAGCTACTCATGCTGACACATTAAGATACAAA
TCCTGGGGCCGTACAGTTATTGGGACAACTGTACCCCATC
CATTAGAAATGTTGGGTCCACAACATCGAAAAGAGACTCC
TTGTGCACCATGTAACACATCAGGGTTCAATTATGTTTCT
GTGCATTGTCCAGACGGGATCCATGACGTCTTTAGTTCAC
GGGGACCATTGCCTGCTTATCTAGGGTCTAAAACATCTGA
ATCTACATCTATTTTGCAGCCTTGGGAAAGGGAAAGCAAA
GTCCCACTGATTAAAAGAGCTACACGTCTTAGAGATGCTA
TCTCTTGGTTTGTTGAACCCGACTCTAAACTAGCAATGAC
TATACTTTCTAACATCCACTCTTTAACAGGCGAAGAATGG
ACCAAAAGGCAGCATGGGTTCAAAAGAACAGGGTCTGCCC
TTCATAGGTTTTCGACATCTCGGATGAGCCATGGTGGGTT
CGCATCTCAGAGCACTGCAGCATTGACCAGGTTGATGGCA
ACTACAGACACCATGAGGGATCTGGGAGATCAGAATTTCG
ACTTTTTATTCCAAGCAACGTTGCTCTATGCTCAAATTAC
CACCACTGTTGCAAGAGACGGATGGATCACCAGTTGTACA
GATCATTATCATATTGCCTGTAAGTCCTGTTTGAGACCCA
TAGAAGAGATCACCCTGGACTCAAGTATGGACTACACGCC
CCCAGATGTATCCCATGTGCTGAAGACATGGAGGAATGGG
GAAGGTTCGTGGGGACAAGAGATAAAACAGATCTATCCTT
TAGAAGGGAATTGGAAGAATTTAGCACCTGCTGAGCAATC
CTATCAAGTCGGCAGATGTATAGGTTTTCTATATGGAGAC
TTGGCGTATAGAAAATCTACTCATGCCGAGGACAGTTCTC
TATTTCCTCTATCTATACAAGGTCGTATTAGAGGTCGAGG
TTTCTTAAAAGGGTTGCTAGACGGATTAATGAGAGCAAGT
TGCTGCCAAGTAATACACCGGAGAAGTCTGGCTCATTTGA
AGAGGCCGGCCAACGCAGTGTACGGAGGTTTGATTTACTT
GATTGATAAATTGAGTGTATCACCTCCATTCCTTTCTCTT
ACTAGATCAGGACCTATTAGAGACGAATTAGAAACGATTC
CCCACAAGATCCCAACCTCCTATCCGACAAGCAACCGTGA
TATGGGGGTGATTGTCAGAAATTACTTCAAATACCAATGC
CGTCTAATTGAAAAGGGAAAATACAGATCACATTATTCAC
AATTATGGTTATTCTCAGATGTCTTATCCATAGACTTCAT
TGGACCATTCTCTATTTCCACCACCCTCTTGCAAATCCTA
TACAAGCCATTTTTATCTGGGAAAGATAAGAATGAGTTGA
GAGAGCTGGCAAATCTTTCTTCATTGCTAAGATCAGGAGA
GGGGTGGGAAGACATACATGTGAAATTCTTCACCAAGGAC
ATATTATTGTGTCCAGAGGAAATCAGACATGCTTGCAAGT
TCGGGATTGCTAAGGATAATAATAAAGACATGAGCTATCC
CCCTTGGGGAAGGGAATCCAGAGGGACAATTACAACAATC
CCTGTTTATTATACGACCACCCCTTACCCAAAGATGCTAG
AGATGCCTCCAAGAATCCAAAATCCCCTGCTGTCCGGAAT
CAGGTTGGGCCAATTACCAACTGGCGCTCATTATAAAATT
CGGAGTATATTACATGGAATGGGAATCCATTACAGGGACT
TCTTGAGTTGTGGAGACGGCTCCGGAGGGATGACTGCTGC
ATTACTACGAGAAAATGTGCATAGCAGAGGAATATTCAAT
AGTCTGTTAGAATTATCAGGGTCAGTCATGCGAGGCGCCT
CTCCTGAGCCCCCCAGTGCCCTAGAAACTTTAGGAGGAGA
TAAATCGAGATGTGTAAATGGTGAAACATGTTGGGAATAT
CCATCTGACTTATGTGACCCAAGGACTTGGGACTATTTCC
TCCGACTCAAAGCAGGCTTGGGGCTTCAAATTGATTTAAT
TGTAATGGATATGGAAGTTCGGGATTCTTCTACTAGCCTG
AAAATTGAGACGAATGTTAGAAATTATGTGCACCGGATTT
TGGATGAGCAAGGAGTTTTAATCTACAAGACTTATGGAAC
ATATATTTGTGAGAGCGAAAAGAATGCAGTAACAATCCTT
GGTCCCATGTTCAAGACGGTCGACTTAGTTCAAACAGAAT
TTAGTAGTTCTCAAACGTCTGAAGTATATATGGTATGTAA
AGGTTTGAAGAAATTAATCGATGAACCCAATCCCGATTGG
TCTTCCATCAATGAATCCTGGAAAAACCTGTACGCATTCC
AGTCATCAGAACAGGAATTTGCCAGAGCAAAGAAGGTTAG
TACATACTTTACCTTGACAGGTATTCCCTCCCAATTCATT
CCTGATCCTTTTGTAAACATTGAGACTATGCTACAAATAT
TCGGAGTACCCACGGGTGTGTCTCATGCGGCTGCCTTAAA
ATCATCTGATAGACCTGCAGATTTATTGACCATTAGCCTT
TTTTATATGGCGATTATATCGTATTATAACATCAATCATA
TCAGAGTAGGACCGATACCTCCGAACCCCCCATCAGATGG
AATTGCACAAAATGTGGGGATCGCTATAACTGGTATAAGC
TTTTGGCTGAGTTTGATGGAGAAAGACATTCCACTATATC
AACAGTGTTTAGCAGTTATCCAGCAATCATTCCCGATTAG
GTGGGAGGCTGTTTCAGTAAAAGGAGGATACAAGCAGAAG
TGGAGTACTAGAGGTGATGGGCTCCCAAAAGATACCCGAA
CTTCAGACTCCTTGGCCCCAATCGGGAACTGGATCAGATC
TCTGGAATTGGTCCGAAACCAAGTTCGTCTAAATCCATTC
AATGAGATCTTGTTCAATCAGCTATGTCGTACAGTGGATA
ATCATTTGAAATGGTCAAATTTGCGAAGAAACACAGGAAT
GATTGAATGGATCAATAGACGAATTTCAAAAGAAGACCGG
TCTATACTGATGTTGAAGAGTGACCTACACGAGGAAAACT
CTTGGAGAGATTAAAAAATCATGAGGAGACTCCAAACTTT
AAGTATGAAAAAAACTTTGATCCTTAAGACCCTCTTGTGG
TTTTTATTTTTTATCTGGTTTTGTGGTCTTCGT

Selective Replication

In house design: Minimal number of basepairs upstream of transcription start site (TSS) that include promoter motifs (TATA-box, initiator, GC-box, CCAAT-box) that is divisible by 3 (to stay in-frame of start codon).

Source: Eukaryotic Promoter Database (EPD) Additional examples of promoters are provided in FIG. 10.

Selective Infection

For retargeting of VSV for CXCR4, the VSV glycoprotein (VSV-G) gene was deleted in the DNA editor, and sequences for measles virus (MeV) fusion (F) and hemagglutinin (H) glycoproteins were uploaded as a replacement. This may be accomplished through diverse viral glycoproteins, RSV, GALV, Reovirus, SeV, among others. The natural affinity of MeV-F/H for receptors CD46 and SLAM was knocked out via mutations Y481A and R533A, respectively. Then VSV-F/H constructs were equipped with the ability to target and infect CXCR4 via one of three mechanisms.

A combination of strategies was used for retargeting/to alter tropism.
1) Other viral glycoproteins that naturally and exclusively target and infect the receptor of interest were expressed.
2) Natural molecules and ligands known to bind the receptor of interest were identified, and the 3D structure was examined via Protein Data Bank (PDB). Binding domain sequences were then fused to the C-terminal end of MeV-H.
3) scFv, bispecific, and other antibody sequences were fused to the C-terminus of the glycoprotein for exclusive binding and internalization

Alternative:

To augment infection specificity, viruses were modified via pseudotyping, glycoproteins were mutated to eliminate host tropism and natural binding affinity, and antibody, ligand, etc., sequences were fused to the C-terminus of the glycoprotein for exclusive binding and internalization of cancer cells. For example, VSV-G was replaced with Measles virus fusion (F) and hemagglutinin (H) glycoproteins, MeV-F/H wildtype sequences were mutated to knock out affinity for CD46, Nectin-4, and SLAM, and then cancer specific binding sequences were fused to MeV-H (e.g. HER2 scFv). This may be accomplished through diverse viral glycoproteins, RSV, MeV, GALV, Reovirus, SeV, among others. Sequence data was obtained from sources including EMBL, Genbank, Genecards, and UniProt.

Digital Virus Identifier (DVI)

To ensure safety and provide a means for automatic identification and data capture, a Digital Virus Identifier (DVI) is incorporated in the genome. This is a standardized genetic barcode that provides information on the virus species, all modifications of source virus, etc. The framework for this system is composed of a text-to-nucleotide converter and a unique numerical identifier/electronic product code with the following format:
1. Unique restriction enzyme cut site
2. Forward stop codon (5′ to 3′)
3. Public database reference/ID for wildtype virus or source genetic material
4. Unique virus
5. Reverse stop codon (3′ to 5′)
6. Restriction enzyme cut site used in Step #1

For example, the framework may include
1. NotI
2. TAG
3. VSV GenBank ID: J02428
4. HGI-007
5. CTA (complementary sequence for TAG)
6. NotI.

An example of a unique genetic watermark is defined by the following sequence:
GCGGCCGCTAGGTTTCTACTAGAACTCGCCACTCATACCTGCCCTCTTCTT ATCTAGCGGCCGC (SEQ ID NO: 1). The sequence data is for modularized VSV (VSV-MOD).

Genome Assembly

The complete genome design was deconstructed into 0.3-1.8 kb fragments, which were manufactured via Twist Bioscience's silicon-based technology platform. Following DNA synthesis, 1000 ng sequence verified gene fragments were resuspended in 100 uL of nuclease-free water (NFW) and amplified via Multiple Displacement Amplification (MDA) with the REPLI-g Whole Genome Amplification kit (Qiagen).

To construct full-length virus genomes, amplified fragments were assembled on ice via homologous recombination. The reaction contained 0.15pmol of mini fragments (<3000 bp), 0.05 pmol of mega fragments (>3000 bp), and commercial DNA assembly master mix at a concentration ranging from 1:10 to 1:2 of the total reaction volume. The reaction tube/plate was vortexed briefly, centrifuged for 10 seconds at 2000G, and run on a thermocycler for 30 minutes at 65C. Samples were cooled to 4C and then used in transfection or stored at −20C.

Transfection/Virus Rescue

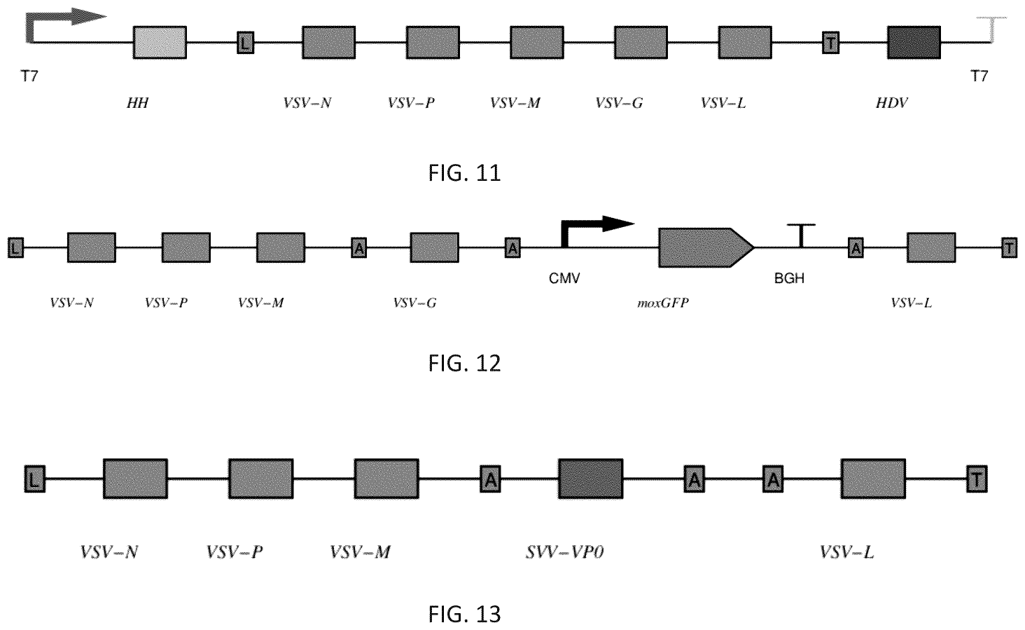

Up to 2 μg of VW support plasmids (VS V-N, VSV-P, VSV-G, VW-L; Kerafast) were linearized by restriction enzyme digestion using Psil-v2 (New England BioLabs). These linear DNA fragments and up to 5 μg of linear DNA encoding full-length genome (VSV or other as described; see FIGS. 11-13) were transcribed in vitro using a T7 expression system (Promega) according to the manufacturer's instructions.

The positive sense mRNA yield was then transfected in BHK-21 using Lipofectamine 3000 (Invitrogen). Cells were seeded in a 6-well plate (Corning) 24 hours prior to transfection at a density of 5×10⁵ cells per well. During transfection, BHK-21 was maintained in DMEM supplemented with 5% EBS. After 24-72 hours, the media was harvested, centrifuged at 300×G for 5 minutes, and the viral supernatant was collected. Cells were then harvested, lysed to release any virus particles, centrifuged at 300×G for 5 minutes to pellet debris, and then the supernatant was collected. Both supernatants were then pooled. After confirming virus rescue, the pool supernatant was amplified.

Alternative

BHK-21 cells were plated in a 12-well plate (Corning) with complete DMEM media. After 24 hours, the cells were washed with PBS (lx) and transfected with full-length VSV genomes using Lipofectamine 3000 (Invitrogen). During this process, the cells were cultured with DMEM with 5% FBS. Virus replication and propagation were confirmed by fluorescence. After 72 hours, virus particles were collected, freeze-thawed with liquid nitrogen (3×), and stored in −80C until needed.

Infection/Amplification

BHK-21 was seeded in a 6-well plate (5×10⁵ cells), T75 flask (2.1×10⁶ cells), or larger vessel, as needed. After 24 hours, cells were infected with synthetic oncolytic viruses rescued from transfection. At 24-72 hours post-infection, the media was harvested, centrifuged at 300×G for 5 minutes, and the viral supernatant was collected. Cells were then harvested, lysed to release any virus particles, centrifuged at 300×G for 5 minutes to pellet debris, and then the supernatant was collected. Both supernatants were then pooled and stored at −80° C. until needed.

Alternative

Cancer cells of interest were plated in 6-, 12-, 24-, 48-, or 96-well plates. After 24 hours, the cells were washed with PBS (1X) and infected with aliquots of virus particles previously collected from transfection. Cells were maintained in FluoroBrite DMEM (Gibco) with 5% FBS at 37C, 5% CO2.

Virus Quantification

Reverse transcription-quantitative polymerase chain reaction (RT-qPCR) was performed for in vitro quantification of vesicular stomatitis virus genomes. Primer and probe mix (FAM labelled) and positive control template for establishing a standard curve were obtained from Primerdesign™ Ltd. VSV (Indiana) positive control template was used for the dilution series with the following copy number: 2×10⁵, 2×10⁴, 2×10³, 2×10², 20, and 2 per μL. Transfection with Lipofectamine 3000 (ThermoFisher) in a 12-well plate (Corning) routinely yielded ˜1×10⁸ VSV genome (equivalents) or copies per mL.

Real-Time Visualization and Analysis

To determine virus growth and kinetics, VSV constructs were studied with mCherry reporter genes via the IncuCyte Live-Cell Analysis system (Sartorius). Following infection, cell culture plates were placed inside the IncuCyte system and maintained at 37C and 5% CO2. Red fluorescence and HD phase contrast images (3-6 per well) were acquired at 10× magnification on an hourly schedule. Cell count and confluence percentage were assessed to monitor cell health and viability. To quantify VSV presence and activity, red object count was recorded for each image, measurements were averaged for each sample/well per timepoint, and then graphed for the duration of the experiment (72 hours in total). In addition, total red object area (m 2/image) and total red object integrated intensity (RCU×μm²/image) were determined; Mean and standard deviation were calculated for each sample/well and graphed over time (hours).

Cytotoxicity Assay

To detect and quantify cell death, reflecting potency and efficacy of viral therapies, cells of interest (e.g. tumor, normal/healthy) were seeded and incubated with IncuCyte Green Cytotoxicity Reagent (Sartorius)+/−synthetic viruses (treatment group). The IncuCyte reagent is a nucleic acid dye that will emit a fluorescent signal after binding DNA in a dying cell with unhealthy/leaky plasma membrane. Similarly, green object count (per image), total green object area (μm²/image), and total green object integrated intensity (RCU×μm²/image) were measured over time (hours). Colocalization of red (mCherry) and green (IncuCyte reagent) fluorescence was indicative of viral presence, replication, and the induction of cell lysis.

Validation of Cell Proliferation and Virus Growth Kinetics

In addition, several other instruments and methods were used to quantify fluorescence and validate results obtained from the IncuCyte system. Among these, GlowMax Discover (Promega), a multimodal microplate reader, was used to measure relative fluorescence units (RFU) and intensity via 520 nm (excitation) and 580-640 nm (emission) filters at serial timepoints (e.g. 0, 3, 6, 24, 48, and/or 72 hours post-infection). Also, EVOS FL Auto (ThermoFisher), an automated cell imaging platform, was used for area scanning, tile stitching, cell counting, and the visualization and analysis of reporter gene expression (e.g. DsRed, mCherry, EGFP).

Results

Generation of Fully Synthetic VSV Virus

A fully synthetic VSV virus was generated having a moxGFP transgene driven by a CMV promoter (see FIG. 12). The virus was rescued and amplified using the methods described above. After amplification images were taken and fluorescence was seen. In addition, a sample was obtained after amplification and flow cytometry was performed.

Leveraging the described T7 promoter and ribozyme system, VSV-WT and VSV-moxGFP were constructed and rescued via transfection methods described herein. Afterwards, BHK-21 was seeded and infected with a known quantity of virus particles (VP). After 48 hours, flow virometry was used to quantify the number of virus particles in the supernatant and the lysate (following 3×freeze-thaw cycles). The increase in the number of VSV and VSV-GFP particles demonstrates that these are replication competent viruses.

TABLE 1
Rescued VSV and VSV-GFP are replication competent.

| | VSV | VSV-GFP |
|---|---|---|
| Cell Line | BHK-21 | BHK-21 |
| Cell Culture Dish/Flask | 100 mm | 100 mm |
| Cells Seeded | 2.2 × 10⁶ | 2.2 × 10⁶ |
| Input (VP) | 82,437 | 88,037 |
| Output: Supernatant (VP) | 9.6 × 10⁶ | 5.9 × 10⁶ |
| Output: Lysate(VP) | 11.3 × 10⁶ | 9.8 × 10⁶ |
| Output: Total(VP) | 20.9 × 10⁶ | 15.8 × 10⁶ |

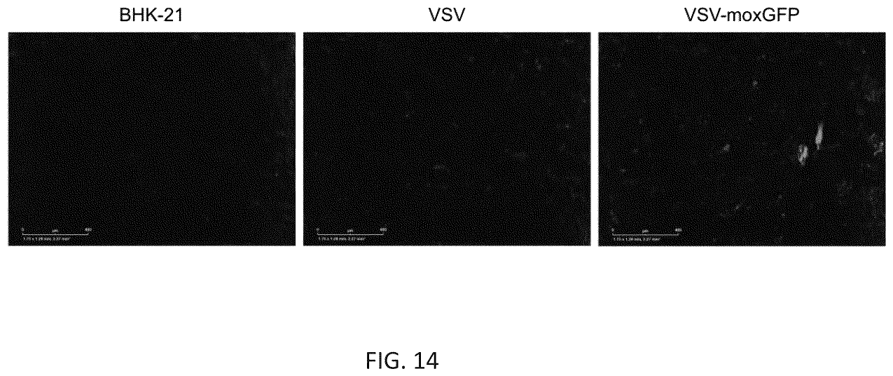

GFP images (10× magnification) depict cells only (control), VSV wildtype, and VSV encoding a moxGFP transgene driven by a CMV promoter at 24 hours post-transfection (FIG. 14). The elevated GFP area and intensity in the VSV-moxGFP image indicate successful transfection and production of GFP (reporter gene). These results demonstrate successful transfection and production of functional VSV virus particles, which validates the modular genome assembly approach. Additionally, the fluorescence activity establishes the capacity to deliver a transgene payload. In alternative aspects, the location in the genome of moxGFP, or modular slot, may be used to load and deliver therapeutic transgenes or genes of interest.

Replacing Glycoproteins in Virus

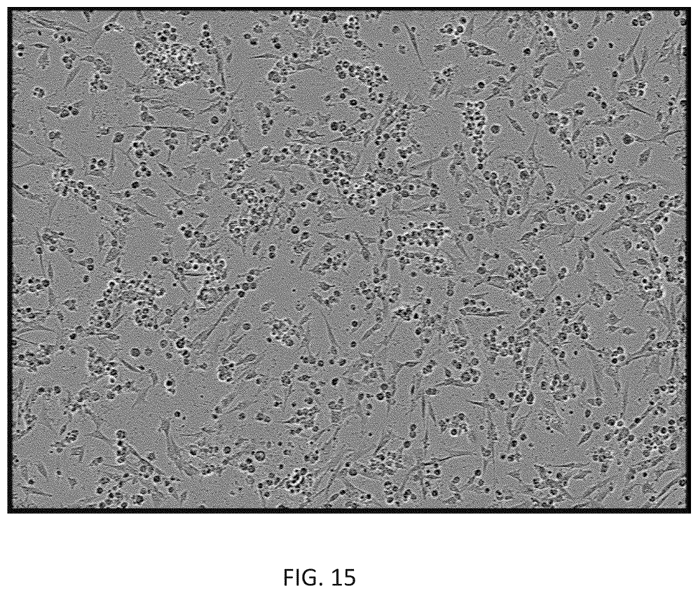

A replicating infectious virus was generated where the glycoproteins of VSV are replaced with the glycoproteins of SVV (see FIG. 13). The virus was rescued and amplified using the methods described above. After amplification images were taken and cytopathic effects were noted. A phase image (10× magnification) of VSV-SVV causing CPE at 24 hours post-transfection in BHK-21 (see FIG. 15).

Engineering Synthetic Promoters

Cell lines of interest (cancer and healthy) were seeded in 48-well plates at a density of 0.03×10⁶ cells per well. After 24 hours, DNA fragments (5×10⁵ fragments total) encoding a promoter of interest and sfGFP reporter were assembled and then transfected using lipofectamine 3000 per the manufacturer's recommendations. After 24 hours, fluorescence intensity was evaluated to validate promoter designs and determine strength.

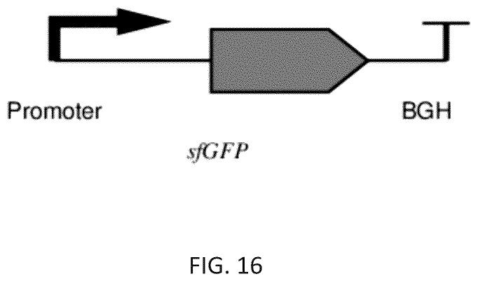

Based on transcriptome data collected from qPCR and RNA-seq for cells of interest, differential gene expression and amplified oncogenes were identified. Putative promoter sequences were then identified, designed, and submitted for DNA synthesis (see FIG. 16).

Canine Synthetic Promoters are Functional and Exhibit Predicable Behavior

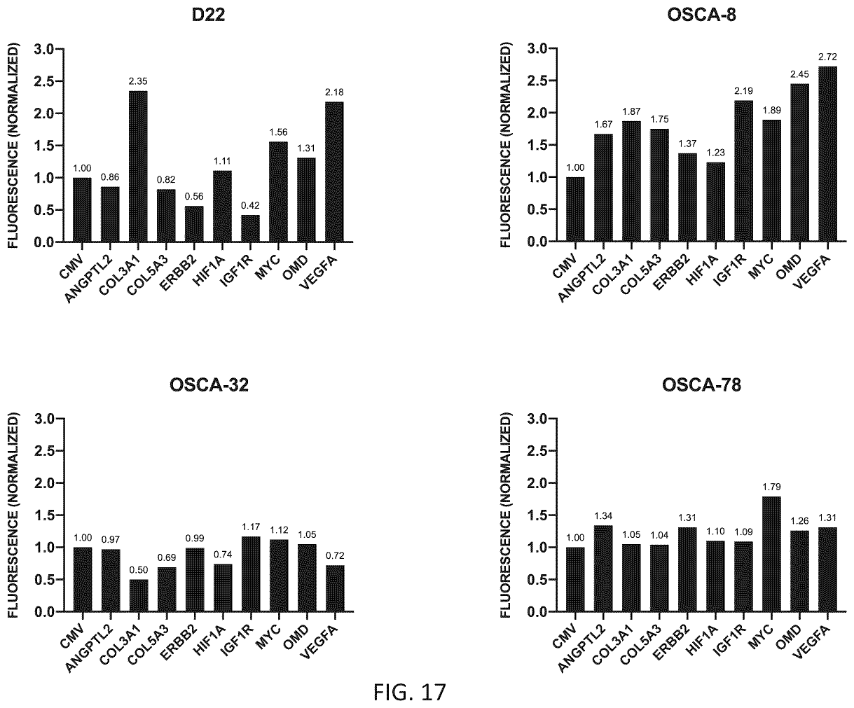

Cell lines of interest (OSCA-8, OSCA-32, OSCA-78, and D22) were seeded in 48-well plates, monitored for 24 hours, and then transfected with DNA fragments encoding a cancer specific promoter and reporter (sfGFP). Putative promoter sequences for COL5A3, VEGFA, ERBB2/HER2, ANGPTL2, OMD, IGF1R, MYC, COL3A1, and HIF1A were designed and included in this experiment. Fluorescence intensity was measured for control wells (cells only) to determine background fluorescence and set GFP threshold gates for analysis. Green fluorescence intensity (a.u.) was then measured and normalized to cell area per image to determine promoter activity. CMV, a promoter well known to the industry, was used as a control to gauge promoter activity and strength. To accomplish this, values were normalized to fluorescent output generated by CMV (see FIG. 17).

Human Synthetic Promoters are Functional and Exhibit Predictable Behavior

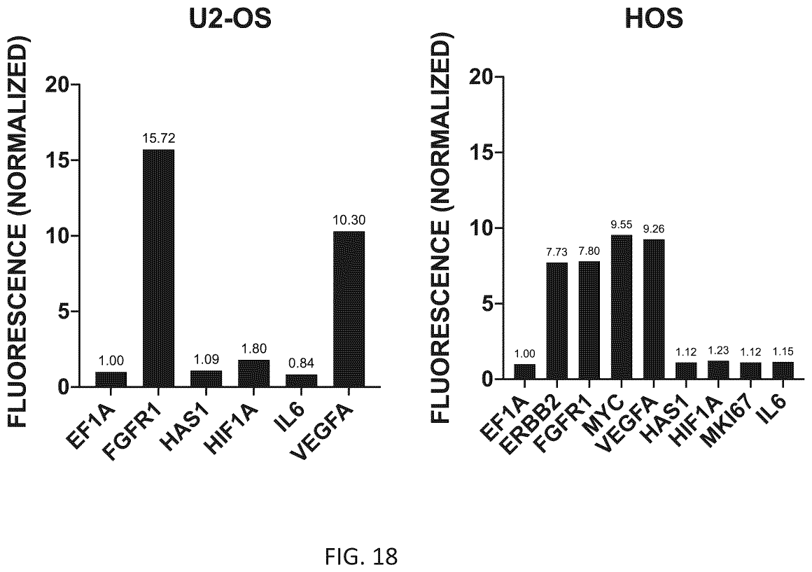

Cell lines of interest (U2-OS, HOS) were seeded in 48-well plates, monitored for 24 hours, and then transfected with DNA fragments encoding a cancer specific promoter and reporter (sfGFP). Human synthetic promoters were evaluated in U2-OS and HOS, including putative sequences for ERBB2/HER2, VEGFA, FGFR1, HAS1, HIF1A, MYC, MKI67, and IL6. Fluorescence intensity was measured for control wells (cells only) to determine background fluorescence and set GFP threshold gates for analysis. Green fluorescence intensity (a.u.) was then measured and normalized to cell area per image to determine promoter activity. EF1A, a well-known, strong mammalian promoter, was used in this experiment as a control promoter to assess strength and activity of putative designs (FIG. 18).

Promoter Designs are Functional and Predictable

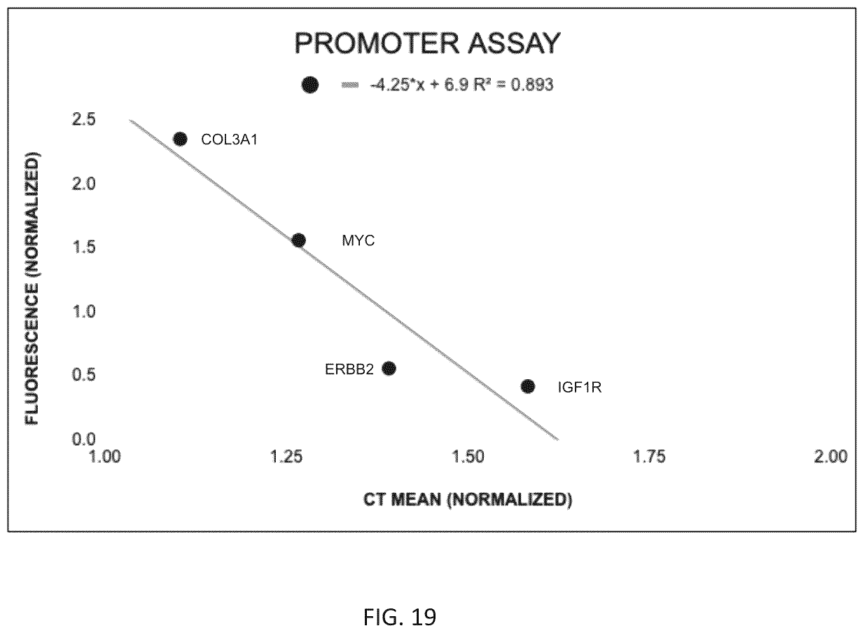

RNA was extracted from D22 using a TaqMan Cells-to-CT kit (Thermo Fisher). Real-time PCR reactions were conducted with Fast Advanced Master Mix (Applied Biosystems) using a QuantStudio 6 Flex (Applied Biosystems). Reaction and cycling conditions were performed according to the manufacturer's recommendations. TaqMan assays (Applied Biosystems) for COL3A1, MYC, ERBB2, and IGF1R were used to determine gene expression. The CT mean for each gene was normalized to a reference gene (GAPDH) activity and plotted on the x-axis. Promoter assay data (fluorescence measurements background subtracted and normalized to CMV) is plotted on the y-axis (FIG. 19). The described design approach successfully generates cancer specific promoters of equal quality.

Cancer Specific Promoters Display High Selectivity

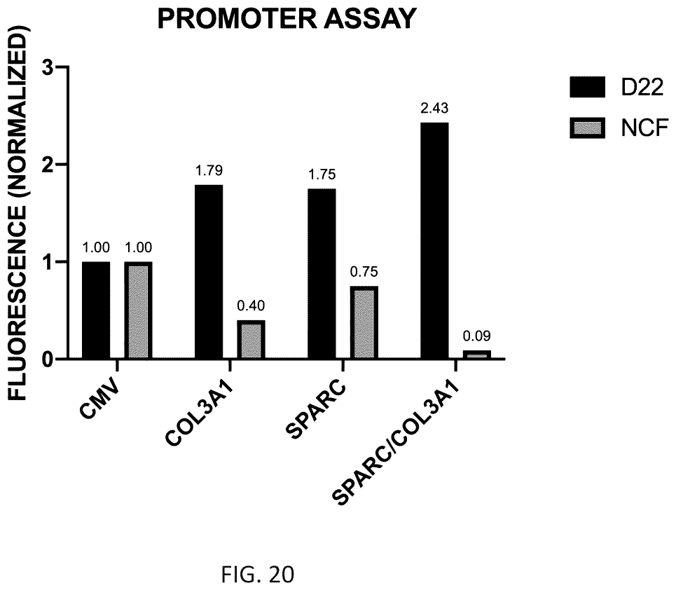

D22 (canine osteosarcoma cell line) and NCF (healthy canine cells) were seeded in a 48-well plate, incubated for 24 hours, and then transfected in triplicate with DNA fragments encoding cancer specific promoters and reporter genes (mKate2). HD Phase and RFP images (n=3 images/well) were acquired at 10× magnification after 24 hours. Fluorescence intensity was measured for control wells (cells only) to determine background fluorescence and set RFP threshold gates for analysis. Red fluorescence intensity (a.u.) was then measured and normalized to cell area per image to determine promoter activity. Values were then normalized to fluorescent output generated by a strong mammalian promoter known to industry—CMV (FIG. 20).

What is claimed is:

1. An artificial oncolytic virus comprising a domain engineered to selectively bind to a target cell via a binding partner identified as highly expressed or preferentially expressed on the target cell as compared with a non-target cell.

2. An artificial oncolytic virus according to claim 1, wherein the binding partner is both highly expressed and preferentially expressed on the target cell as compared with a non-target cell.

3. An artificial oncolytic virus according to claim 1, wherein expression of the viral genome is engineered to be under the control of a regulatory region of a gene identified as highly expressed or preferentially expressed by the target cell as compared with a non-target cell.

4. An artificial oncolytic virus according to claim 3, wherein the gene is identified as both highly expressed and preferentially expressed by the target cell as compared with a non-target cell.

5. An artificial oncolytic virus according to any of claims 3-4, wherein the gene is specifically expressed by the target cell.

6. An artificial oncolytic virus according to any of claims 1-5, wherein the binding partner is specifically expressed by the target cell.

7. An artificial oncolytic virus according to any of claims 1-6, wherein the domain is encoded by a heterologous DNA sequence.

8. An artificial oncolytic virus according to any of claims 1-7, wherein the domain comprises all or a functional portion of a glycoprotein.

9. An artificial oncolytic virus wherein expression of the viral genome is engineered to be under the control of a regulatory region of a gene identified as highly expressed or preferentially expressed by the target cell as compared with a non-target cell.

10. An artificial oncolytic virus according to claim 9, wherein the gene is identified as both highly expressed and preferentially expressed by the target cell as compared with a non-target cell.

11. An artificial oncolytic virus according to any of claims 1-10, wherein the target cell is a cancer cell.

12. An artificial oncolytic virus according to any of the claims 1-10, wherein the target cell is a cell associated with a hyperproliferative disorder.

13. An artificial oncolytic virus according to any of claims 1-10, wherein the target cell is a tumor cell.

14. An artificial oncolytic virus according to any of claims 1-13, wherein the virus is non-pathogenic in a subject to whom it is intended to be administered.

15. An artificial oncolytic virus according to any of claims 1-14, wherein the virus is one to which a subject to whom it is intended to be administered is not immune.

16. An artificial oncolytic virus according to any of claims 1-15, wherein the virus is substantially identical to a reference naturally-occurring virus.

17. An artificial oncolytic virus according to any of claims 1-15, wherein the virus comprises at least one domain which is identical to a reference naturally-occurring virus.

18. An artificial oncolytic virus according to any of claims 1-8 and 11-17, wherein the virus is identical to a reference naturally-occurring virus except for the engineered domain.

19. An artificial oncolytic virus according to any of claims 3-17, wherein the virus is identical to a reference naturally-occurring virus except for the engineered regulatory region.

20. An artificial oncolytic virus according to any of claims 16-19, wherein the reference naturally-occurring virus is a member of the Rhabdoviridae family.

21. An artificial oncolytic virus according to any of claims 16-19, wherein the reference naturally-occurring virus is from the genus Vesiculovirus.

22. An artificial oncolytic virus according to any of claims 16-19, wherein the reference naturally-occurring virus is Indiana vesiculovirus.

23. An artificial oncolytic virus according to any of claims 16-19, wherein the reference naturally-occurring virus is New Jersey vesiculovirus.

24. An artificial oncolytic virus according to any of claims 14-15, wherein the subject is Canis lupus familiaris.

25. An artificial oncolytic virus according to any of claims 14-15, wherein the subject is Homo sapiens.

26. An artificial oncolytic virus according to any of claims 1-25, wherein when administered to a subject said virus causes cell death via apoptosis, necrosis, and/or cytopathic effect (CPE) of one or more target cells in said subject.

27. An artificial oncolytic virus according to claim 26, wherein cell death occurs within 28 days, preferably within 21 days, more preferably within 7 days, more preferably within 2-4 days.

28. An artificial oncolytic virus according to any of claims 1-27, wherein the target cell is selected from the group consisting of cells from the bladder, blood, bone, bone marrow, brain, breast, colon, esophagus, gastrointestine, gum, head, kidney, liver, lung, nasopharynx, neck, ovary, prostate, skin, stomach, testis, tongue, and uterus.

29. An artificial oncolytic virus according to any of claims 1-27, wherein the target cell a cancer cell and wherein the cancer is selected from the group consisting of melanoma, non-small cell lung cancer, small-cell lung cancer, lung cancer, hepatocarcinoma, retinoblastoma, astrocytoma, glioblastoma, gum cancer, tongue cancer, leukemia, neuroblastoma, head cancer, neck cancer, breast cancer, pancreatic cancer, prostate cancer, renal cancer, bone cancer, testicular cancer, ovarian cancer, mesothelioma, cervical cancer, gastrointestinal cancer, lymphoma, brain cancer, colon cancer, and bladder cancer.

30. An artificial oncolytic virus according to any of claims 1-8 and 11-17, wherein the binding partner is identified using RNAseq data.

31. An artificial oncolytic virus according to any of claims 3-17, wherein the gene is identified using RNAseq data.

32. An artificial oncolytic virus according to any of claims 30-31, wherein the RNAseq data is obtained from samples from a group of individuals.

33. An artificial oncolytic virus according to any of claims 30-31, wherein the RNAseq data is obtained from a database.

34. An artificial oncolytic virus according to any of claims 30-31, wherein the RNAseq data is obtained from an individual subject who is the intended recipient of said virus.

35. An artificial oncolytic virus according to any of claims 1-34, wherein said virus additionally comprises one or more heterologous functional domains wherein said functional domain is selected from the group consisting of a therapeutic agent, a kill switch for said target cell, an agent which facilitates the ability of the virus to evade the recipient immune system, a watermark, a barcode, an agent which degrades the extracellular matrix of a solid tumor, and a diagnostic agent.

36. An artificial oncolytic virus according to claim 35, wherein the one or more heterologous functional domains are inserted in frame.

37. An artificial oncolytic virus comprising a sequence of SEQ ID NO: 2.

38. A pharmaceutical composition comprising an artificial oncolytic virus comprising a domain engineered to selectively bind to a target cell via a binding partner identified as highly expressed or preferentially expressed on the target cell as compared with a non-target cell.

39. A method of treating a hyperproliferative disorder in a subject comprising administering to the subject a pharmaceutical composition comprising an effective amount of an artificial oncolytic virus comprising a domain engineered to selectively bind to a target cell via a binding partner identified as highly expressed or preferentially expressed on the target cell as compared with a non-target cell.

40. A method according to claim 39, wherein the hyperproliferative disorder is cancer.

41. A method according to claim 40, wherein the cancer is selected from the group consisting of melanoma, non-small cell lung cancer, small-cell lung cancer, lung cancer, hepatocarcinoma, retinoblastoma, astrocytoma, glioblastoma, gum cancer, tongue cancer, leukemia, neuroblastoma, head cancer, neck cancer, breast cancer, pancreatic cancer, prostate cancer, renal cancer, bone cancer, testicular cancer, ovarian cancer, mesothelioma, cervical cancer, gastrointestinal cancer, lymphoma, brain cancer, colon cancer, and bladder cancer.

42. A method according to any of claims 39-41, wherein the subject is Canis lupus familiaris.

43. A method according to any of claims 39-41, wherein the subject is Homo sapiens.

44. A method of slowing, inhibiting or reducing the growth or size of a tumor comprising administering to the subject a pharmaceutical composition comprising an effective amount of an artificial oncolytic virus comprising a domain engineered to selectively bind to a target cell via a binding partner identified as highly expressed or preferentially expressed on the target cell as compared with a non-target cell.

45. A method according to claim 44, wherein the tumor is selected from the group consisting of non-small cell lung cancer, small-cell lung cancer, lung cancer, hepatocarcinoma, retinoblastoma, astrocytoma, glioblastoma, neuroblastoma, head cancer, neck cancer, breast cancer, pancreatic cancer, prostate cancer, renal cancer, bone cancer, testicular cancer, ovarian cancer, mesothelioma, cervical cancer, gastrointestinal cancer, brain cancer, colon cancer, and bladder cancer.

46. A method according to any of claims 44-45, wherein the subject is Canis lupus familiaris.

47. A method according to any of claims 44-45, wherein the subject is Homo sapiens.

48. A method of producing an artificial oncolytic virus comprising:
(a) designing the DNA for said virus to produce viral DNA, wherein the DNA comprises a domain engineered to selectively bind to a target cell via a binding partner identified as highly expressed or preferentially expressed on the target cell as compared with a non-target cell;
(b) defining fragments of the viral DNA for synthesis;
(c) synthesizing the viral DNA fragments;
(d) assembling the viral DNA fragments to create artificial oncolytic viral DNA; and (e) transfecting cells with said artificial oncolytic viral DNA to produce artificial oncolytic virus.


SEQUENCE LISTING (2 sequences)

SEQ ID NO: 1 - DNA, 64 bp, Artificial
  watermark
gcggccgctaggtttctactagaactcgccactcatacctgccctcttcttatctagcggccgc

SEQ ID NO: 2 - DNA, 11353 bp, Artificial
  modularized vesicular stomatitis virus
acgaagacaaacaaaccattattatcattaaaaggctcaggagaaactttaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggtttgtatgcacaggatccagaagctgagcaagttgaaggctttatacaggggcctttagatgactatgcagatgaggaagtggatgttgtatttacttcggactggaaaccacctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacactcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctaacttctagcttctgaacaatccccggtttactcagtctctcctaattccagcctctcgaacaactaatatcctgtcttttctatccctatgaaaaaaattttcatagattcaactgttttcatagtaaaaccaacgtaactaagcttcatcccaatagtgctaatactaatgccgtcaactgtttgctctaacagagatcgatctgtttccttgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccatacaagtcaaaatgcccaagagtcacaaggctattcaagcagacggttggatgtgtcatgcttccaaatgggtcactacttgtgatttccgctggtatggaccgaagtatataacacagtccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaattttccctataaataggccgtctaatattttagtgcttttaactagcataataaacaatacaacgtttgctgtgctacataggccgtctagtctagctaattctaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggactatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaacttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgt